(19) United States
(12) Patent Application Publication
(10) Pub. No.: US 2010/0087374 A1
(43) Pub. Date: Apr. 8, 2010

(54) Methods for Treatment and Diagnosis of Pulmonary Diseases Based on the Expression of SERCA2 Protein

(76) Inventors: Shama Ahmad, Aurora, CO (US); Carl W. White, Denver, CO (US)

Correspondence Address: SHERIDAN ROSS PC, 1560 BROADWAY, SUITE 1200, DENVER, CO, 80202, US

(21) Appl. No.: 12/573,605

(22) Filed: Oct. 5, 2009

Related U.S. Application Data
(60) Provisional application No. 61/102,805, filed on Oct. 3, 2008.

(51) Int. Cl. A61K 38/16; A61K 31/7052; A61K 31/56; A61K 31/135; A61K 31/44; G01N 33/68; G01N 33/48; A61P 11/00

(52) U.S. Cl. 514/12; 514/44 R; 514/169; 514/653; 514/342; 436/86; 436/94

(57) ABSTRACT

The present invention is directed to methods of treatment of cystic fibrosis. The invention includes a method for treatment of cystic fibrosis in a patient by increasing the activity of sarcoendoplasmic reticulum calcium ATPase (SERCA) in a patient. More specifically, the step of increasing SERCA activity can include but is not limited to, administration of SERCA protein or its homologues, gene therapy to restore or enhance SERCA activity, or the administration of compounds stimulating the activity of endogenous SERCA. Reference herein to SERCA, can include in preferred embodiments, the isoform SERCA2, which is the principal lung isoform of SERCA. The present invention is based on the finding that SERCA2 (a calcium pump) is deficient (not 100%) in the lung epithelial cells of cystic fibrosis samples.

### CROSS-REFERENCE TO RELATED APPLICATIONS

This application claims the benefit of priority under 35 U.S.C. §119(e) from U.S. Provisional Application Ser. No. 61/102,805, filed Oct. 3, 2008, the contents of which are incorporated herein in their entirety by this reference.

### GOVERNMENT SUPPORT

This invention was supported in part with funding provided by NIH Grant No. R01-ES014448 awarded by the National Institute of Health. The government has certain rights to this invention.

### FIELD OF THE INVENTION

The field of the present invention is methods for treatment and diagnosis of pulmonary diseases such as Cystic Fibrosis (CF) using SERCA2 protein.

### BACKGROUND OF THE INVENTION

Cystic fibrosis (CF) is a genetic disorder known to be an inherited disease of the secretory glands. While life expectancies have increased to nearly 40 years, respiratory failure still accounts for >80% of deaths from the disease, usually in young adults in the third or fourth decade of life. CF is caused by a mutation in the gene cystic fibrosis transmembrane conductance regulator (CFTR). The product of this gene is a chloride ion channel important in creating sweat, digestive juices and mucus. Although most people without CF have two working copies (alleles) of the CFTR gene, only one is needed to prevent cystic fibrosis. CF develops when neither allele can produce a functional CFTR protein. Therefore, CF is considered an autosomal recessive disease.

Relentless progressive lung infection and excessive secondary inflammation remains the principal cause of death in CF. Airways of urban dwellers are continuously bombarded by atmospheric pollutants such as ozone, particulates and nitrogen oxides. Elevated levels of such pollutants can contribute to repeated exacerbations and thereby accelerated decline of lung function in patients with chronic airway disease like cystic fibrosis (CF) and asthma (71-73). Ozone attacks the lung through oxidative mechanisms and causes disruption of the epithelial barrier leading to increased permeability, an influx of neutrophils into the lungs, and generation of cytokines and chemokines (74-77). CF airways have enhanced oxidative stress as evidenced by elevated levels of products of lipid peroxidation in the exhaled breath condensate and biofluid samples. Excessive depletion of airway antioxidants as well as malnutrition further add susceptibility to potential oxidant injury. Acute exacerbations interrupt the clinical course of CF and hasten decline of lung function. Knowledge of the role of environmental pollutants like ozone, in this process is limited. Identification of mechanisms leading to pulmonary exacerbations in the patient with CF is crucial for developing therapies for maintenance of lung function, good quality of life and survival.

As noted above, Ca²⁺-dependent Cl⁻ secretion and Na⁺ absorption are two primary known ion transport mechanisms that are affected in CF airway epithelial cells. Regulation of epithelial ion transport in airway epithelia is of great importance for continuous adjustment of surface mucus hydration. Maintenance of a constant thickness of the surface water/mucus layer is critical for function of the mucociliary clearance mechanism, which removes inhaled particles from the airways. Investigations of endogenous regulatory mechanisms have demonstrated that nucleotides (ATP and UTP) are released to the surface layer and could be involved in regulation of ion transport by acting as paracrine or autocrine agents through interaction with purinergic receptors on the epithelial surface. The mechanism(s) by which nucleotides are released to the surface layer is not known in detail, but some results (78-80) indicate that the cystic fibrosis transmembrane conductance regulator (CFTR) protein may be involved as a modulator. ATP signaling may regulate numerous critical cell functions relevant to CF including volume homeostatic responses to altered tonicity of the extracellular milieu, ciliary beat frequency, epithelial cell secretion of ions, fluid and, mucin secretion, and release of inflammatory cytokines (81). CFTR-mediated nucleotide release within airway surface liquid (ASL) regulates epithelial ion transport rates by Ca²⁺- and protein kinase C-dependent mechanisms (82, 83). Such regulation of intracellular calcium [Ca²⁺]i by CFTR may in turn normalize NaCl transport in CF airway epithelia by stimulating Ca²⁺-dependent Cl⁻ secretion (84) and simultaneously downregulating Na⁺ hyperabsorption (85). However, mutations in CFTR affect its ability to be made, processed, and transported to the plasma membrane and/or to function as a Cl⁻ channel and conductance regulator. Ca²⁺ signaling finely controls survival and secretory function of the airway epithelium, processes that are altered in CF, although Ca²⁺ signaling also may itself be altered by CF.

The most frequent CF-associated mutation, accounting for about 70% of CF alleles, is deletion of phenylalanine 508 (ΔF508 CFTR). ΔF508 CFTR has reduced chloride channel activity, impaired processing, and decreased stability at the cell surface. Abnormal processing leads to its retention in the endoplasmic reticulum (ER) and rapid intracellular degradation. For these reasons, ΔF508 CFTR fails to function as a cAMP-activated Cl⁻ channel (1).

Previous reports have indicated that sarcoendoplasmic reticulum calcium ATPase (SERCA) inhibitors can decrease calcium concentrations within the ER and, thereby, interfere with the ability of calcium-dependent chaperone proteins to retain misfolded ΔF508 CFTR within the ER (2). These investigators have suggested further that blockade of this chaperone interaction by use of SERCA inhibitors allows misfolded ΔF508 CFTR to escape the ER, reach the cell surface, and function as a Cl⁻ channel (3). These findings precipitated a remarkable number of investigations and resulted in several conflicting papers indicating that SERCA pump inhibitors like curcumin and/or thapsigargin can (4-9) or cannot (10-13) enhance ΔF508 CFTR trafficking to the plasma membrane and apical epithelial chloride transport.

Release of calcium ions from ER regulates essential cellular functions including secretion, contraction, gene transcription, and survival (14). Since SERCA is responsible for (re) loading of ER calcium after such signaling events, its function can be important at the level of the whole organ and organism. For example, SERCA2 is the only SERCA isoform expressed in cardiac muscle, and decreased SERCA2 expression is a key event in congestive heart failure (15). Its importance is further demonstrated by the lethal phenotype of SERCA2 knockout mice and loss of calcium regulation in cells with only one copy of the SERCA2 gene (16, 17). In CF, release of ER calcium by activation of purinergic receptors can allow activation of Ca²⁺-activated chloride channels (CACC), a principal, albeit partial, compensatory mechanism for impaired chloride secretion in CF that could, in turn, somewhat diminish excessive sodium reabsorption by ENaC (18). Hence, ER Ca²⁺ stores can have direct importance in adaptation of CF epithelium.

However, despite the research into potential effects of SERCA inhibitors on ΔF508 CFTR trafficking and function, the expression of SERCA isoforms in CF and non-CF airway epithelium has not been systematically evaluated. Further, role of SERC in the survival of airway epithelium in response to oxidative stress is also not well understood. The present application addresses these needs in the art and proposes therapeutic and diagnostic methods based on the findings described herein.

### SUMMARY OF THE INVENTION

In one embodiment, the present invention includes a method to treat a pulmonary disease in a subject. The method may comprise increasing the biological activity of Sarcoendoplasmic Reticulum Calcium ATPase 2 (SERCA2) protein in the cells of the subject.

In another embodiment, the present invention includes a method to protect a subject from exposure to an oxidizing gas. The method may comprise increasing the biological activity of SERCA2 protein in the cells of the subject. In some embodiments, the subject may have a pulmonary disease and exposure to the oxidizing gas may lead to enhanced airway epithelial cell death and inflammation leading to exacerbation of the pulmonary disease. In some embodiments, the oxidizing gas may comprise ozone, oxygen, chlorine or mustard gas.

In some embodiments, the methods of the present invention may include the step of administering the subject with an effective amount of an agent that increases the biological activity of the SERCA2 protein. The agent may comprise a SERCA2 protein or a homologue thereof, or a compound that increases the expression of the SERCA2 protein, or a SERCA2 activator compound that increases the biological activity of the SERCA2 protein. In some embodiments, the SERCA2 protein or a homologue thereof is recombinantly produced. In some embodiments, the compound that increases the expression of the SERCA2 protein may comprise a recombinant nucleic acid molecule encoding the SERCA2 protein or a homologue thereof. 21. In some embodiments, the recombinant nucleic acid molecule encoding the SERCA2 protein or a homologue thereof may comprise a sequence selected from the group consisting of: NM_{—}170665.3 or GI:161377445, NM_{—}001681.3 or GI:161377446, and NM_{—}001135765.1 or GI:209413708). In some embodiments, the SERCA2 protein or a homologue thereof may comprise an amino acid sequence selected from the group consisting of NP_{—}733765.1 or GI:24638454, NP_{—}001672.1 or GI:4502285, and NP_{—}001129237.1, or GI:209413709. In some embodiments, the SERCA2 activator compound may comprise PST2744 [Istaroxime; (E,Z)-3-((2-aminoethoxy)imino) androstane-6,17-dione hydrochloride)], Memnopeptide A, JTV-519, CDN1054, albuterol, xopenex, IGF (insulin like growth factor), EGF (epithelial growth factor), or rosiglitazone. In some embodiments, the agent may comprise a pharmaceutically acceptable carrier. In some embodiments, the step of administering comprises providing the agent as a tablet, a powder, an effervescent tablet, an effervescent powder, a capsule, a liquid, a suspension, a granule or a syrup.

In another embodiment, the present invention includes a method for diagnosing a pulmonary disease. In some embodiments, the method comprises detecting a level of expression or biological activity of the SERCA2 protein in a test sample, and comparing the level of expression or biological activity of the SERCA2 protein in the test sample to a baseline level of SERCA2 protein expression or activity established from a control sample, wherein detection of a statistically significant difference in the SERCA2 protein expression or biological activity in the test sample, as compared to the baseline level of SERCA2 protein expression or biological activity, is an indicator of the presence of the pulmonary disease or the potential therefor in the test sample as compared to cells in the control sample. In various embodiments, detecting the level of expression or biological activity of the SERCA2 protein in a sample may comprise detecting SERCA2 mRNA in the sample, or detecting SERCA2 protein in the sample, or detecting SERCA2 protein biological activity in the sample

In another embodiment, the present invention includes a method to evaluate the efficacy of a treatment of a pulmonary disease in a subject. The method may comprise the steps of detecting the level of expression or biological activity of SERCA2 in a test sample taken from the subject before administering the treatment; detecting the level of expression or biological activity of SERCA2 in a test sample taken from the subject after administering the treatment; and comparing the level of the expression or biological activity of the SERCA2 in the test sample taken from the subject before administering the treatment to the level of the expression or biological activity of the SERCA2 in the test sample taken from the subject after administering the treatment. In various embodiments, detecting the level of expression or biological activity of SERCA2 in a test sample may comprise detecting SERCA2 mRNA in the test sample, or detecting SERCA2 protein in the test sample, or detecting biological activity of the SERCA2 protein in the test sample.

In some preferred embodiments, the SERCA2 protein may be expressed in airway epithelial cells. In some embodiments, the subject is human. In some embodiments, the pulmonary disease is Cystic Fibrosis.

### BRIEF DESCRIPTION OF THE DRAWINGS

FIG. 1 shows SERCA2 protein and RNA expression in non-CF and CF cell lines. 1A and 1B show representative Western (experiments repeated 6 times) and Northern blots (experiments repeated 3 times), respectively. FIG. 1C shows co-immunostaining for of SERCA2 (red) and endoplasmic reticulum (ER) specific protein, protein disulphide isomerase (PDI, green) (one data set from an experiment performed in duplicate. The individual experiment was repeated 3 times).

FIG. 2 shows estimation of endoplasmic reticulum (ER) content in FIG. 2A and SERCA2 (protein and activity) in purified microsomal membranes FIG. 2B. The upper panel of FIG. 2A shows live cells cultured in chambered coverglass stained using ER-Tracker Blue-White DPX. The lower panel of FIG. 2A shows the quantification of fluorescence intensity per whole cells area. For each of 3 cell lines about 20 cells were analysed. Results show means of data and * indicates significant difference (p<0.05) from non-CF 16HBEo- cells (n=3). The top panel in FIG. 2B is a representative Western blot showing SERCA2 expression in purified microsomal membranes of 16HBEo- (lane 1), CF4lo- (lane 2) and CF45o- (lane 3) cells. The lower panel of FIG. 2B represents the thapsigargin (2 μM)-sensitive Ca²⁺ ATPase activity in microsomal membranes of normal and CF cells. * indicates significant difference from 16HBEo- cells (p<0.05).

FIG. 3 shows SERCA2 expression in air-liquid interface (ALI) cultures of primary non-CF and CF airway epithelial cells. FIG. 3A shows representative images of in situ immunohistochemistry for SERCA2 expression in cells from 4 individual (1-4) non-CF donors and 4 individual (5-8) CF donors. FIG. 3B is a representative Western blot showing expression of SERCA2 and actin in the cells from four non-CF (1-4) and four CF (5-8) individuals. FIG. 3C shows quantification of Western blots for SERCA2 expression in ALI cultures of cells from non-CF and CF donors (14 non-CF and 8 CF) analysed in 3 separate experiments. The bars represent means of data and * indicates significant difference (p<0.05) from non-CF cells.

FIG. 4 shows SERCA2 expression in the epithelium of proximal and distal CF and non-CF airways. FIGS. 4A-D show the IgG staining (left panels) and SERCA2 staining (right panels) in epithelium. Arrowheads [symbol] indicate SERCA2 staining which was found predominantly in the epithelium of non-CF bronchi (A) and bronchioles (C), and it was significantly less intense in the epithelium of CF airways (B & D). FIG. 4E shows the quantitation of SERCA2 staining (SERCA2-IgG) in the non-CF and CF bronchi and FIG. 4F shows quantitation of SERCA2 staining in the non-CF and CF bronchioles.

FIG. 5 shows expression of low affinity SERCA isoform SERCA3 in CF and non-CF airway epithelial cells. FIG. 5A, top row, represents the Western blot for SERCA3 from whole cell lysates from non-CF and CF bronchial airway epithelial cell lines. FIG. 5B represents the Northern blot for SERCA3 mRNA expression. FIG. 5C represents the Western blot for SERCA3 from cell lysates from ALI cultures of primary airway epithelial cells from 3 non-CF (1-3) and 3 CF (4-6) subjects.

FIG. 6 shows the effect of CFTRᵢₙₕ172 on SERCA2 protein expression. FIG. 6A shows SERCA2 expression in CFTRᵢₙₕ172-treated primary bronchial epithelial cells (Lane 1-3 are untreated control & 4-6 are CFTRᵢₙₕ172-treated cells). FIG. 6B shows the quantitative data for SERCA2 expression with and without CFTRᵢₙₕ172 treatment. The bars represent means of data and * indicates significant difference (p<0.05) from non-CF cells (results of 3 individual experiments are shown). FIG. 6C is a representative blot showing effect of CFTRᵢₙₕ172 on SERCA2 expression treatment in CF IB3-1 cells and CFTR corrected C-38 cells. FIG. 6D shows the quantitative data. The bars represent mean of data and * indicates significant difference (p<0.05) from untreated cells n=3.

FIG. 7 shows the effect of inhibition of functional CFTR expression on SERCA expression. FIG. 7A shows inhibition by antisense CFTR oligonucleotides. The upper panel is a representative Western blot showing SERCA2 expression in cell lysates from polarized cultures of 16HBEo- cell line that were stably transfected with sense (S) and antisense (AS) CFTR oligonucleotide. The bars represent means of data and * indicates significant difference (p<0.05) from control (16HBE-S) cells. FIG. 7B shows the effect of overexpressing mutated CFTR on SERCA2 expression. The upper panel is a representative Western Blot showing expression of SERCA2 in control cells and adenovirally-transduced minimally transformed primary human bronchial epithelial cells expressing mutated ΔCFTR.

FIG. 8 shows translocation of SERCA2 within caveolae-related domains (CRDs) from the ER of CF cells. FIG. 8A shows the distribution of CRD-associated proteins within a sucrose gradient, and representative Western blots showing SERCA2 and caveolin expression. FIGS. 8B and 8C show Western blot of SERCA2 and Bcl-2 using immunoprecipitate of microsomal fractions from (1) 16HBEo- and (2) CF45o- using Bcl-2 antibody.

FIG. 9 shows that Bcl-2 expression increased in the cellular compartments of CF cells. FIG. 9A shows representative Western blots using antibodies against Bcl-2, cytochrome c oxidase (mitochondrial marker), protein disulphide isomerase (PDI, ER specific protein) and lamin C (nuclear marker) to analyze nuclear, ER and mitochondrial fractions from (1) 16HBEo-, (2) CF4lo- and (3) CF45o- cells. FIG. 9B shows Bcl-2 expression in the ER fraction of 16HBEo- cell line stably transfected with sense (S) and antisense (AS) CFTR oligonucleotide. FIG. 9C shows total Bcl-2 content in cellular lysates of control and adenovirally-transduced primary human bronchial epithelial cells expressing mutated ΔCFTR. The bars represent means of data of two individual experiments (n=4) and * indicates significant difference (p<0.05) from control.

FIG. 10 shows that SERCA2 is essential for cell survival during oxidative stress. FIG. 10A shows SERCA2 knockdown using SERCA2 siRNA. The top panel of FIG. 10A is the representative Western blot of SERCA2 expression in primary human bronchial epithelial cells either (1) mock-transfected or transfected with (2) control or (3) SERCA2 siRNA. The lower panel shows the quantitation of SERCA2 knockdown. The bars represent means of data and * indicates significant difference (p<0.05) from control siRNA-transfected cells (n=3), and represents 3 individual experiments. FIG. 10B shows the cell death analysis in primary human airway epithelial cells that were transfected either with a control or SERCA2 siRNA for 24 h and exposed to 0 ppb or 200 ppb ozone for 18 h. The columns represent means of data and * indicates significant difference (p<0.05) from 0 ppb controls cells. # indicates significant difference from 200 ppb controls (n=3).

FIG. 11 shows the effect of CFTR inhibition on ATP release and cell survival of primary human bronchial epithelial cells in ozone. Primary human bronchial epithelial cells cultured on collagen-coated 6-well plates were treated with CFTRᵢₙₕ172 for 30 min and then exposed to either 0 ppb (−) or 200 ppb (+) ozone. FIG. 11A shows the ATP content of the extracellular media and FIG. 11B shows cell death was estimated after 8 h of ozone exposure. The bars represent means (SEM) of data and * indicates significant difference (p<0.05) from 0 ppb (− ozone) controls and # indicates significant difference (p<0.05) from 200 ppb exposed cells without CFTRᵢₙₕ172.

FIG. 12 shows the extracellular ATP release by non-CF and CF airway epithelial cell air liquid interface (ALI) cultures upon ozone exposure. FIG. 12A shows ATP content of culture media from ozone exposed non-CF 16HBE (open bars) and CF, CF4lo- (closed bars) and CF45o- (hatched bars). The bars represent means of data and * indicates significant difference (p<0.05) from 0 ppb controls and # indicates significant difference (p<0.05) from 200 ppb exposed non-CF cells. FIG. 12B shows quantification of apical ATP release in differentiated ALI cultures of primary cells from non-CF and CF donors (3 non-CF and 3 CF) analysed in 3 separate experiments (The mean of the non-CF group is the control value). The bars represent means of data and * indicates significant difference (p<0.05) from 0 ppb control and # indicates significant difference (p<0.05) from ozone exposed non-CF cells.

FIG. 13 shows Ozone-induced membrane damage in non-CF and CF cells. FIGS. 13A and 13B show ³H-adenine release in the culture media from ALI cultures of 16HBE and CF45o- cells labeled with ³H-adenine and exposed to ozone. The bars represent means (SEM) (mean of non-CF represents the control) of data of apical media and * indicates significant difference (p<0.05) from 0 ppb. FIG. 13C shows Calcien AM (green, live) and propidium iodide (PI) (red, dead) cellular staining of ALI cultures of non-CF, 16 HBE and CF, CF4lo- and CF45o- cells that were exposed to either 0 or 500 ppb ozone. FIG. 13D shows quantitation of dead PI +ve cells. The bars represent means (SEM) of data and * indicates significant difference (p<0.05) from 0 ppb.

FIG. 14 shows ozone-mediated apoptosis in non-CF (open bars) and CF (closed bars) cells. The bars represent means (SEM) of data and * indicates significant difference (p<0.05) from 0 ppb and # indicates significant difference (p<0.05) from non-CF.

FIG. 15 shows the effect of ozone exposure on mitochondrial function of non-CF and CF cells. The top panel shows Measurement of chloromethyltetramethylrosamine fluorescence, an indicator of mitochondrial membrane potential (MMP) in 16HBEo- (open bars) and CF45o- (closed bars) cells that were exposed to ozone FIGS. 15A and 15B shows representative immunocytochemistry images showing cytochrome c release in 16HBEo- and CF4lo- cells exposed to ozone.

FIG. 16 shows the ozone-induced ERK phosphorylation in non-CF and CF cells. FIG. 16A shows a representative Western blot for the detection of ERK phosphorylation by Western blot (top panel). FIG. 16B shows a quantitative estimation of total ERK phosphorylation in non-CF (open bars) and CF cells (closed bars). The bars represent means (SEM) of data and * indicates significant difference (p<0.05) from 0 ppb.

FIG. 17 shows the effect of ozone exposure on the release of proinflammatory cytokines IL-8, G-CSF and GM-CSF, in differentiated cultures of non-CF (, 0 ppb and ◯, 200 ppb) and CF (▴, 0 ppb and Δ, 200 ppb) primary airway epithelial cells. FIGS. 17 A and 17B show the analysis of cytokines in the apical and basolateral media, respectively. The bars represent means (SEM) of data and # indicates significant difference (p<0.05) from 0 ppb exposed cells and * indicates significant difference (p<0.05) from 200 ppb non-CF, using Welch's t test.

FIG. 18 shows the Ozone-mediated cytokine release in polarized air-liquid interface cultures of non-CF (16HBEo) and CF (CF4lo) cell lines. FIGS. 18A, 18B and 18C show release of IL-8, G-CSF or GM-CSF respectively from non-CF (open bars) and CF (closed bars). Values are means±SE; n=6, and the figure is a representative of 4 individual experiments; * Significant difference from 0 ppb control, P<0.05 and # Significant difference from non-CF, P<0.05. FIG. 18D shows the effect of preincubation of cells for 30 min with 10 μM [6-amino-4-(4-phenoxyphenylethylamino)quinazoline] (an NF-κB inhibitor) on IL-8 release. FIG. 18E shows the measurement of nuclear p65 in non-CF and CF cells after exposure to ozone. Values are means±SE; * Significant difference from 0 ppb control, P<0.05 and # Significant difference from non-CF, P<0.05.

FIG. 19 shows the effect of ATP supplementation on ozone-mediated cytokine release. FIGS. 19A and 19B show the release of L-8 and GM-CSF in the apical media. * Significant difference from 0 ppb control, P<0.05 and # Significant difference from non-CF, P<0.05.

FIG. 20 shows that SERCA2 modulates cytokine release by primary airway epithelial cells. FIG. 20A shows the release of IL-8 in the supernatant media from cells preincubated with thapsigargin and then exposed to ozone. Values are means±SE; n=6 and the data is a representative of 2 individual experiments; * Significant difference from 0 ppb control, P<0.05 and # Significant difference from ozone exposed untreated cells, P<0.05. FIG. 20B shows release of IL-8 from cells that were transduced with Ad.GFP or Ad.SERCA2 and then exposed to ozone. The inset is a representative Western blot showing SERCA2 protein overexpression by Ad.SERCA2 in primary airway epithelial cells. Values are means±SE; n=6 and the data is a representative of 2 individual experiments; * Significant difference from 0 ppb control, P<0.05 and # Significant difference from 200 ppb ozone exposed Ad.GFP transduced cells, P<0.05.

FIG. 21 is a schematic representation of mechanisms of ozone toxicity in CF airway epithelial cells.

FIG. 22A shows the CFTR function in CFTR sense and antisense oligonucleotide-expressing 16HBEo- cells indicating that the cAMP-dependent chloride conductance was absent in CFTR antisense oligonucleotides expressing cells. FIG. 22B is a representative blot showing the CFTR expression in adenovirally transduced primary airway epithelial cells.

FIG. 23 shows mutated CFTR-dependent increases in NF-κB and Bcl-2 in primary human airway epithelial cells in the presence and absence of TNF. FIG. 23, top panel shows Western blot of CFTR expression in lacz, WT CFTR and mutated CFTR expressing cells. Lower panel shows Western blot of p65 in the nuclear lysate. FIG. 23B shows quantification of nuclear translocation of NF-κB as measured by ELISA. The bars represent mean of data and * indicates significant difference (p<0.05) from untreated cells. FIG. 23C is a Western blot for Bcl-2 in whole cell lysates.

### DETAILED DESCRIPTION

The present invention relates to methods of treatment and diagnosis of pulmonary diseases, such as Cystic Fibrosis (CF). As described in detail herein, the expression of the SERCA2 protein is decreased in the airways of lungs of subjects having cystic fibrosis as compared to the non-CF subjects. Specifically, expression of the principal lung isoform, SERCA2, was consistently downregulated in CF airways, primary cultures of polarized CF airway epithelial cells, in response to genetic or pharmacologic inhibition of wild-type CFTR expression or function, and upon expression of ΔF508 CFTR in non-CF epithelium. Even though the low affinity SERCA3 isoform was upregulated, total SERCA activity was decreased. In CF cells, SERCA2 was associated with Bcl-2 in ER membranes. SERCA/Bcl-2 interaction has previously been reported to cause inactivation and displacement of SERCA from membrane microdomains referred to as caveolae-related domains (CRD) (19, 20). Therefore, without wishing to be bound by theory, it is proposed that CFTR dysfunction-induced enhanced NF-κB activation, and subsequently increased Bcl-2 that interacted with SERCA2 to translocate it from the caveolae-related domains (CRDs), were potential mechanisms causing decreased SERCA2 expression.

Furthermore, Knockdown of SERCA2 using siRNA revealed that it is required for survival of airway epithelial cells during oxidative stress. Diminished SERCA2 expression enhanced apoptosis and/or cell death due to oxidative stress indicating that SERCA2 was essential for survival during oxidative stress.

An association between exposure to oxidizing gases like ozone and exacerbations of preexisting pulmonary diseases such as asthma and chronic obstructive pulmonary disease has not been previously evaluated in CF. In the present invention, effects of ambient concentrations of ozone on human primary non-CF and CF airway epithelial cells and cell lines cultured at air liquid interface were investigated. Ozone toxicity was determined by measuring transepithelial resistance, ³H adenine release and fluorescent live/dead cell staining on the inserts. As described in detail herein, exposure to ozone of polarized cultures of CF airway epithelial cells caused enhanced loss of transepithelial resistance, release of ³H adenine and death. Ozone exposure caused enhanced loss of mitochondrial membrane potential, release of cytochrome c and apoptosis in CF airway epithelial cells. Ozone exposure also caused enhanced proinflammatory cytokine (IL-8 and GM-CSF) production in CF airway epithelial cells. Release of extracellular ATP upon ozone exposure was diminished in CF cells, and supplementation of purine nucleotides enhanced ozone-mediated cytokine release. These studies demonstrate that ozone exposure may cause enhanced airway epithelial cell death and inflammation leading to exacerbation of CF disease.

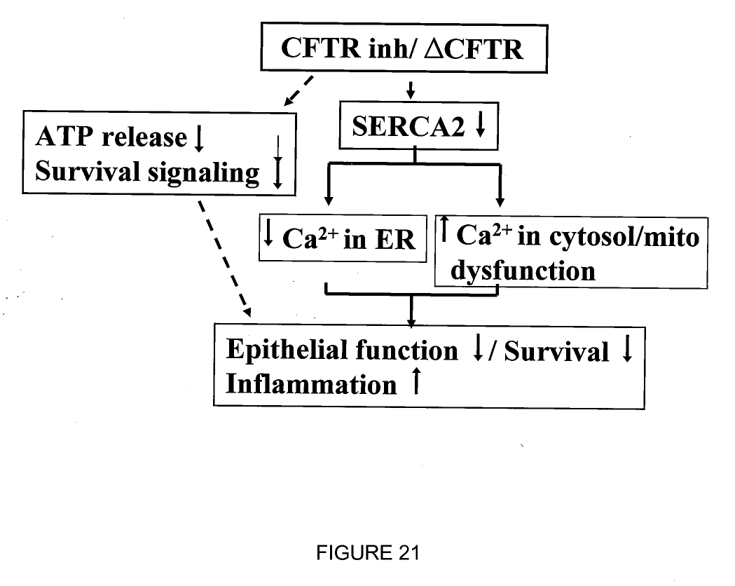

The ozone-induced enhanced inflammation in CF airways is a consequence of decreased SERCA activity. As described in examples below, inhibition of SERCA caused enhanced IL-8 release and overexpression of SERCA2 decreased ozone-mediated cytokine production. (Modulation of IL-8 by SERCA2 overexpression was unknown prior to this invention.) This would also suggest that CF airway inflammation maintains cytosolic calcium levels by decreasing SERCA expression and slowing Ca²⁺ reuptake (FIG. 21). Therefore, modulation of SERCA2 would prove to be a useful strategy for treating CF inflammation or controlling exacerbations due to environmental pollutants like ozone.

As described in detail in the examples below, the present invention on the expression of SERCA2 in CF airways relative to expression in respiratory cells and tissues of non-CF individuals revealed that SERCA2 protein expression is decreased in CF airway epithelial cell lines and primary polarized airway epithelial cells grown at air-liquid interface (ALI) as well as in vivo in distal and proximal airway epithelial tissue of CF subjects. CFTR dysfunction-induced enhanced NF-κB activation, and subsequently increased Bcl-2 that interacted with SERCA2 to translocate it from the caveolae-related domains (CRDs), were potential mechanisms causing decreased SERCA2 expression. In the series of experiments involving primary cells grown at ALI, although there was considerable variability in expression from donor to donor, the overall extent of SERCA2 protein expression was diminished in primary CF cells. Individual variability in the primary cells could have resulted from variations in genotype, types of differentiated cells present (mucus, ciliated, etc), the types and/or severity of infection(s) previously present, presence of additional disease states (e.g. diabetes etc) in the donor (38), and/or types of medications previously administered (39). Also of note, SERCA2 protein expression was quantitatively diminished, as detected by immunohistochemistry, in epithelial cells of large and smaller airways in lungs of CF patients. This supports the clinical relevance of these findings.

Previous studies indicated that altered calcium homeostasis in airway epithelial cells of CF patients is mainly a result of inflammation and infection present (40, 41). The airway epithelial cell lines utilized in the present studies were maintained in the absence of infection and inflammation for months to years, and the primary cells examined were cultured in the absence of infection and inflammatory cells for 1-6 weeks. Despite the differences in cell environment, decreased SERCA2 expression was a constant feature of CF airway epithelium, suggesting that it is an intrinsic feature of the disease. This was further supported by IHC studies using lung tissues of CF patients. These tissue samples were certainly not free of infection and chronic inflammation.

ER plays an important role in CF disease pathogenesis. Decreased SERCA2 expression was not due to a diminished ER mass in CF cells. In fact, as indicated by staining for both the ER-specific protein PDI and an ER-specific fluorescent dye, CF airway epithelial cells contained similar or greater quantities of ER. These findings are generally in agreement with previous reports of expanded ER in CF (40). Although abnormal ΔF508 CFTR can be both retained and degraded in ER, and transient adenoviral overexpression of ΔF508 CFTR was sufficient to cause decreased SERCA2 expression in non-CF airway epithelial cells, the findings in our study (e.g. antisense CFTR oligonucleotide expression) do not indicate that presence of mutant CFTR(s) in the ER is the principal cause of diminished SERCA2 expression in CF airway epithelial cells.

Recent investigations on the modulation of SERCA2 activity have reported the presence of an important interaction between SERCA2 and Bcl-2, resulting in SERCA2's translocation from CRD to membrane domains of higher density, partial unfolding, and inactivation (19, 20). Bcl-2 is capable of interaction with SERCA1 or SERCA2. In purified sarcoplasmic reticulum preparations, addition of Bcl-2 causes significant loss of SERCA activity (20). Because of relevant reports in CF (42, 43), the present inventors determined if such an interaction occurs in CF airway epithelium. Bcl-2 protein expression and association with SERCA2 was elevated in CF relative to non-CF airway epithelial cells. Given that Bcl-2 binding to SERCA2 in other tissues can displace it from CRD and inactivate it, the findings reported herein suggest that similar mechanisms also could act in lung epithelium to diminish SERCA2 protein expression and activity in the ER.

A previous study in relation to CF and Bcl-2 expression indicated that Bcl-2 expression increased mucus cell metaplasia of airway epithelial cells and that insufflation of bacterial endotoxin could cause such changes in airways of rats (42). The present studies of this invention CF and non-CF airway epithelial cell lines, and of CFTR sense and antisense oligonucleotide-expressing cells, again, were done in the absence of bacterial infection, inflammation and endotoxin, and these cells were maintained in the absence of these factors for extended periods of time. In addition, the cells in these particular experiments were not cultured at air-liquid interface. Thus, unlike the present studies of primary cell cultures grown at ALI, they could not undergo differentiation to mucus-expressing cells and could not have had altered Bcl-2 expression in association with mucus cell differentiation. In this case, such culture conditions were considered advantageous as they prevented potential Bcl-2 changes due to differentiated phenotype. It is conceivable that some variability in SERCA2 expression in CF primary airway epithelial cells from different donors could have related to differences in mucus cell differentiation of such cultures, but this could not have occurred in the various transformed CF and non-CF cell lines the present inventors studied. Based on the absence of infection, inflammation, bacterial endotoxin and mucus cell differentiation in our experiments pertinent to Bcl-2, the findings of the present invention indicate that increased Bcl-2 expression is an inherent feature of CF airways epithelium.

CFTR dysfunction is associated with aberrations in a number of signaling pathways including those related to inflammation and infection. The specific link(s) between CFTR dysfunction and increased NF-κB activation in CF cells remains undefined. However, there are several factors that could cause this to occur (43). The present data with overexpression of mutant CFTR provides further evidence that CFTR dysfunction causes increased NF-κB activation/p65 mobilization to nucleus. Increased NF-κB-mediated proinflammatory gene transcription has been reported before (24) in the cells expressing CFTR antisense oligonucleotide (16HBE-AS), which is another model studied in the present invention. NF-κB is a transcriptional regulator of Bcl-2 and other proteins of Bcl-2 family (35, 44). Thus, increased Bcl-2 in cells with CFTR dysfunction could result, at least in part, from enhanced NF-κB activation.

Increased expression of Bcl-2 in CF airway epithelium could have beneficial and/or detrimental effects. It could increase proliferation and diminish cell death via its anti-apoptotic action. Increased proliferation in submucosal gland and basal cells of CF airways has been described before (45), and this could have an important role in airway epithelial regeneration and repair. Further, osmotic challenges routinely faced by CF airway epithelial cells might be better tolerated in presence of Bcl-2 overexpression. CF epithelial cells have increased susceptibility to osmotic stress and blunted regulatory volume decrease (RVD) responses (46, 47), and Bcl-2 overexpression can stimulate such responses and promote survival (48). Moreover, by inhibiting apoptosis in the presence of oxidative stress and/or infection, Bcl-2 expression at high levels could favor necrosis of airway epithelium and enhance release of cell contents like DNA and proteolytic enzymes, worsening airways damage and obstruction. Such effects of Bcl-2 may be site-specific. In this context and that of the present study, it is noteworthy that others recently described a paradoxical pro-apoptotic effect of high level Bcl-2 expression when Bcl-2 is localized to the nucleus (49). Although Bcl-2 expression often acts to oppose apoptosis and cell death, it might do otherwise in the context of CF, if, for example, its effects on SERCA2 secondarily alter other functions, like alternate chloride channels, that could themselves influence cell fate.

Members of the Bcl-2 family regulate ER Ca²⁺ homeostasis. Bcl-x(L) binds to the inositol trisphosphate receptor (InsP(3)R) Ca²⁺ release channel to enhance Ca²⁺ release resulting in reduced ER [Ca²⁺], increased oscillations of cytoplasmic Ca²⁺ concentration ([Ca²⁺]ᵢ), and resistance to apoptosis (50). Bcl-2 also increases emptying of endoplasmic reticulum Ca²⁺ stores during photodynamic therapy-induced apoptosis (51). Emptying of ER stores following SERCA inhibition with thapsigargin causes cells to undergo growth arrest or apoptotic cell death (52). Thus, Bcl-2 and Bcl-2 related proteins regulate apoptosis by altering ER [Ca²⁺] via modulation of InsP(3)R and/or SERCAs (20, 53). However, it is not clear whether reduced ER [Ca²⁺] or enhanced [Ca²⁺]ᵢ signaling is most relevant for apoptosis protection.

Although the literature is mixed on the ability of CF cells to undergo apoptosis, recent reports indicated that CF airway epithelial cells are more prone to apoptosis and/or necrosis than non-CF cells (54, 55). Despite the anti-apoptotic effects that Bcl-2 could have in the CF airway, the present inventors sought to define whether downregulation of SERCA2 in airway epithelial cells, whether via Bcl-2 or other mechanisms, impacts cell survival there. Specifically, it was investigated herein whether diminished SERCA2 expression could enhance apoptosis and/or cell death. Primary airway epithelial cells expressing SERCA2 siRNA showed enhanced toxicity to three different stimuli, each of which can contribute, directly and indirectly, to oxidative stress in CF airways (56). These included ozone, hydrogen peroxide, and TNFα+IL-1β. The present invention indicated the capacity of SERCA2 downregulation to modulate susceptibility to cell death due to oxidative stress.

There are multiple examples in the cardiovascular literature in which SERCA modification can potentially affect the disease process. Oxidative stress may modify SERCA structure and/or function (57). In CF airways, oxidative stress may be innate and/or secondary to inflammation. Cholesterol also can affect SERCA expression (58). Indeed, cholesterol also may accumulate excessively in CF lung (59). In one model, heterozygous SERCA2 knockout (SERCA2a^{+/-}) mice showed substantially enlarged infarction after cardiac ischemia (60). In addition, they showed impaired postischemic myocardial relaxation and reduced postischemic myocardial contractile function. Further, these animals had both higher diastolic intracellular calcium without oxidative stress, as well as higher intracellular calcium following the oxidative stress of reperfusion. The level of reactive oxygen species production was not increased in the SERCA2 knockouts, but the level of dysfunction related to oxidative stress was increased. Thus, there are precedents for substantial alterations in cell survival and function in the heart with comparable changes in SERCA2 expression to those seen in CF, relative to non-CF, lung cells.

Intracellular calcium signaling depends upon SERCA activity. [Ca²⁺]ᵢ can regulate a number of important functions pertinent to airway epithelial cells, including mucus secretion, ciliary contraction and motility, signal transduction, and cell survival. Diminished SERCA activity in CF could dampen such intracellular calcium signals and blunt adaptive chloride export. Novel purinergic compounds in development for treatment of CF are directed at stimulating intracellular calcium signals by acting on extracellular ATP receptors that activate this process (61). Notably, it was recently reported that ATP-induced increased short circuit currents were lower in CF than non-CF small airway epithelial cell cultures (62). Taken together, these data suggest that SERCA inhibitor therapy would not likely be beneficial in CF, and, further, that SERCA inhibition may worsen airway epithelial cell survival under oxidative stress and decrease alternative calcium-dependent chloride transport. Hence, an alternate approach using SERCA-stimulating interventions in the treatment of CF is provided by the present invention.

The present findings indicate that the previously proposed strategy of SERCA inhibition would not be of benefit in CF. Instead, contrary to the previous view in the field, increasing the activity or expression of SERCA2 expression in airway cells would provide important therapeutic benefits. While the data presented herein is based on the model of Cystic Fibrosis, the findings reported herein are applicable to pulmonary diseases in general and increasing the activity of SERCA2 would provide therapeutic benefits in a wide range of pulmonary diseases. Examples of diseases where the methods of present invention are useful include, without limitation, asthma, Chronic obstructive pulmonary disease (COPD), chronic bronchitis, non-CF bronchiectasis and primary ciliary dyskinesia (PCD).

SERCA or Sarco-endoplasmic Reticulum Ca²⁺ ATPase, is a calcium pump which transfers Ca⁺² from the cytosol of the cell to the lumen of the SR. The SERCA proteins are encoded by a family of three genes SERCA1, SERCA2 and SERCA3 that are highly conserved but are localized on different chromosomes. The nucleotide sequences of these genes are well known in the art. (Genomics. 1993 August; 17(2):507-9. Chromosome mapping of five human cardiac and skeletal muscle sarcoplasmic reticulum protein genes. Otsu K, Fujii J, Periasamy M, Difilippantonio M, Uppender M, Ward D C, MacLennan D H.) The SERCA isoform diversity is dramatically enhanced by alternative splicing of the transcripts and multiple isoforms od SERCA, such as SERCA1a,b, SERCA2a-c, and SERCA3a-f, have been detected. These are well described in the art and are known to one skilled in the art. The terms SERCA2, as used herein, refer to all SERCA2 isoforms encoded by the SERCA2 gene.

The nucleotide sequence of the SERCA2 gene is known in the art and is available as GeneID: 488, all the information associated with which is incorporated herein by reference. The Genbank accession numbers for the SERCA2 isoforms a, b and c are available respectively, as NM_{—}170665.3, GI:161377445 (nucleotide) and NP_{—}733765.1, GI:24638454 (protein); NM_{—}001681.3, GI:161377446 (nucleotide) and NP_{—}001672.1, GI:4502285 (protein), and NM_{—}001135765.1, GI:209413708 (nucleotide) and NP_{—}001129237.1, GI:209413709 (protein). All of these sequences are incorporated herein by reference in their entirety.

Further, the term, SERCA2 protein, may also refer to proteins encoded by allelic variants, including naturally occurring allelic variants of nucleic acid molecules known to encode SERCA2 protein, that have similar, but not identical, nucleic acid sequences to naturally occurring, or wild-type, SERCA2-encoding nucleic acid sequences. An allelic variant is a gene that occurs at essentially the same locus (or loci) in the genome as a SERCA2 protein gene, but which, due to natural variations caused by, for example, mutation or recombination, has a similar but not identical sequence. Allelic variants typically encode proteins having similar activity to that of the protein encoded by the gene to which they are being compared. Allelic variants can also comprise alterations in the 5′ or 3′ untranslated regions of the gene (e.g., in regulatory control regions).

According to the present invention, a subject may include any member of the vertebrate class, Mammalia, including, without limitation, primates, rodents, livestock and domestic pets. A preferred subject includes a human, a rodent, a monkey, a sheep, a pig, a cat, a dog and a horse. An even more preferred subject is a human.

The methods of the present invention provide therapeutic benefit in the treatment of a pulmonary disease. As such, a therapeutic benefit is not necessarily a cure for a particular disease or condition, but rather, preferably encompasses a result which most typically includes alleviation of the disease or condition or increased survival, elimination of the disease or condition, reduction of a symptom associated with the disease or condition, prevention or alleviation of a secondary disease or condition resulting from the occurrence of a primary disease or condition, and/or prevention of the disease or condition.

As used herein, the phrase “to treat a pulmonary disease” refers to reducing the potential for a pulmonary disease; reducing the occurrence of the disease, and/or reducing the severity of the disease, preferably, to an extent that the subject no longer suffers discomfort and/or altered function due to it. For example, treating can refer to the ability of a compound, when administered to a subject, to prevent a disease from occurring and/or to cure or to alleviate disease symptoms, signs or causes. The term, “disease” refers to any deviation from the normal health of a mammal and includes a state when disease symptoms are present, as well as conditions in which a deviation (e.g., infection, gene mutation, genetic defect, etc.) has occurred, but symptoms are not yet manifested.

In one embodiment, the present invention includes a method to protect a subject from exposure to an oxidizing gas. As used herein, the phrase “to protect from” a condition or disease refers to reducing the symptoms of the condition or disease; reducing the occurrence of the condition or disease, and/or reducing the severity of the condition or disease. Protecting a subject can refer to the ability of a composition of the present invention, when administered to a subject, to prevent a condition or disease from occurring and/or to cure or to alleviate disease symptoms, signs or causes. As such, to protect a subject from a disease includes both preventing the condition or disease occurrence (prophylactic treatment) and treating a subject that has a disease (therapeutic treatment). A beneficial effect can easily be assessed by one of ordinary skill in the art and/or by a trained clinician who is treating the subject.

As described herein, when a subject has a pre-existing pulmonary disease, exposure to an oxidizing gas can lead to exacerbation of the disease. This may be due an enhanced apoptosis and/or cell death, and inflammation in the lung cells. Examples of an oxidizing gas include, without limitation, ozone, mustard gas and chlorine. The oxidizing gas may also be oxygen. The situations in which the oxidizing gas may be oxygen include situations where a high amount of oxygen is given therapeutically. In a preferred embodiment the oxidizing gas may be ozone.

In some embodiments, the pulmonary disease may be asthma, Chronic obstructive pulmonary disease (COPD), chronic bronchitis, non-CF bronchiectasis and primary ciliary dyskinesia (PCD). In a preferred embodiment, it is cystic fibrosis.

In some embodiments, the method of the present invention includes a step of increasing the biological activity of the SERCA2 protein in the cells of a subject. The subject has, or is at risk of developing, a pulmonary disease. The cells referred herein include lung cells that normally express the SERCA2 protein. Examples of such cells may include, without limitation, epithelial cells which includes multiple epithelial cell types known in the art, and smooth muscle cells. The cells may further include those around airways and blood vessels. In some embodiments, cells to be targeted may be airway (nasal & tracheal/bronchial) epithelial cells. In further embodiments, cells to be targeted may be endothelial cells, smooth muscle cells and neutrophils. These may also include stem cells or progenitor cells isolated from the airways. In a preferred embodiment, the cells are airway epithelial cells.

An increase in SERCA2 biological activity is defined herein as any measurable (detectable) increase (i.e., upregulation, stimulation, enhancement) of the activity of the SERCA2. As used herein, to increase SERCA2 biological activity refers to any measurable increase in SERCA2 biological activity by any suitable method of measurement. The SERCA2 activity may be defined in terms of rate of Ca⁺² transport. Methods of measuring SERCA2 activity are well known in the art. For example, SERCA activity may be determined using the calcium sensitive dye Fluo-4/AM as described before (Cell Calcium, 2005 March; 37(3):251-8, Elevated Ca²⁺(i) transients induced by trimethyltin chloride in HeLa cells: types and levels of response, Florea A M, Dopp E, Büsselberg D), using the highthrough put assay kit from Molecular probes (Invitrogen, Carlsbad Calif.). Briefly, media is removed from the top of cells cultured to confluence in a 96-well plate and replaced with 100 μl 1.0 μM Fluo-4 dye in assay buffer. The plate is incubated for 30 min at 37° C. and then at room temperature for 30 min. Fluoresence (Ex496 nm/Em 516 nm) is then recorded using a fluorescent plate reader (BioTEK, Winooski, Vt.) equipped with 488 argon laser. Typically extracellular ATP (50-100 M) is used as a stimulator of cytosolic calcium and buffer was control.

Increasing SERCA2 biological activity can be accomplished by administering to the subject an agent that increases the total biological activity of the SERCA2 protein in the cells of the subject. In some embodiments the agent may comprise a SERCA2 protein or a homologue thereof. It may also be a synthetic homologue or mimetic. It may be a naturally occurring SERCA2 protein that is obtained by purification from animal tissue. Additionally, it may be a recombinant protein.

In some embodiments, the agent may comprise a compound that increases the expression of or overexpresses the SERCA2 protein in the cells. Overexpression of SERCA2 refers to an increase in expression of the SERCA2 over a normal, endogenous level of SERCA2 expression. For cell types which express detectable levels of the SERCA2 under normal conditions, an overexpression is any statistically significant increase in expression of the SERCA2 (p<0.05) (or constitutive expression where expression is normally not constitutive) over endogenous levels of expression.

The increase in expression or overexpression may be achieved by increasing the transcription and/or the translation of the endogenous SERCA2 gene. In one aspect of this embodiment, the SERCA2 can be effectively overexpressed in a cell by increasing the activity of a promoter for the SERCA2 gene in the cell such that expression of endogenous SERCA2 in the cell is increased. In such embodiments, the agent may comprise a compound that is a transcriptional activator of the SERCA2 gene.

Another method by which SERCA2 overexpression can be achieved is by transfecting the cells with a recombinant nucleic acid molecule encoding the SERCA2 protein, wherein the recombinant SERCA2 is expressed by the cell. Thus, the agent may comprise a recombinant nucleic acid molecule that is capable of encoding the SERCA2 protein or a homologue thereof. Suitable vectors and methods of transfection of cells are well known in the art. Transfection of a nucleic acid molecule according to the present invention can be accomplished by any method by which a nucleic acid molecule can be introduced into the cell in vivo, and includes, but is not limited to, transfection, electroporation, microinjection, lipofection, adsorption, viral infection, naked DNA injection and protoplast fusion.

Preferably, a recombinant nucleic acid molecule is produced using recombinant DNA technology (e.g., polymerase chain reaction (PCR) amplification, cloning). Suitable nucleic acid sequences encoding the SERCA2 for use in a recombinant nucleic acid molecule of the present invention include any nucleic acid sequence that encodes the SERCA2 protein having biological activity and suitable for use in the target host cell. For example, when the target host cell is a human cell, human SERCA2-encoding nucleic acid sequences are preferably used, although the present invention is not limited to strict use of naturally occurring sequences or same-species sequences.

A recombinant nucleic acid molecule includes a recombinant vector comprising the isolated nucleic acid molecule encoding a SERCA2 protein, operatively linked to a transcription control sequence. The phrase “operatively linked” refers to linking a nucleic acid molecule to a transcription control sequence in a manner such that the molecule is expressed when transfected (i.e., transformed, transduced or transfected) into a host cell. Transcription control sequences are sequences that control the initiation, elongation, and termination of transcription. The vector may further comprise translation control sequences, origins of replication, and other regulatory sequences that are compatible with the host cell, which is capable of enabling recombinant production of the SERCA2 protein and of delivering the nucleic acid molecule into the host cell.

Such a vector may contain nucleic acid sequences that are not naturally found adjacent to the isolated nucleic acid molecules to be inserted into the vector. The vector may be either RNA or DNA, either prokaryotic or eukaryotic. In some embodiments, the vector may be a virus or a plasmid. Recombinant vectors can be used in the cloning, sequencing, and/or otherwise manipulating of nucleic acid molecules. Recombinant vectors are preferably used in the expression of nucleic acid molecules, and can also be referred to as expression vectors.

Particularly important transcription control sequences are those that control transcription initiation, such as promoter, enhancer, operator and repressor sequences. Suitable transcription control sequences include any transcription control sequence that can function in a host cell according to the present invention. A variety of suitable transcription control sequences are known to those skilled in the art. Preferred transcription control sequences include those which function in the subject's cells, with cell- or tissue-specific transcription control sequences being particularly preferred. Examples of transcription control sequences include, but are not limited to, transcription control sequences useful for expression of a protein in airway epithelial cells and the naturally occurring SERCA2 promoter. Transcription control sequences may include inducible promoters, cell-specific promoters, tissue-specific promoters and enhancers. Suitable promoters for these and other cell types will be easily determined by those of skill in the art. Transcription control sequences of the present invention can also include naturally occurring transcription control sequences naturally associated with the protein to be expressed prior to isolation.

The recombinant nucleic acid molecule encoding a SERCA2 protein or homologe thereof, may include a recombinant viral vector. Such a vector includes a recombinant nucleic acid sequence encoding a SERCA2 protein of the present invention that is packaged in a viral coat that can be expressed in a host cell in an animal or ex vivo after administration. A number of recombinant viral vectors can be used, including, but not limited to, those based on alphaviruses, poxviruses, adenoviruses, herpesviruses, lentiviruses, adeno-associated viruses and retroviruses. Viral vectors suitable for gene delivery are well known in the art and can be selected by the skilled artisan for use in the present invention. A detailed discussion of current viral vectors is provided in “Molecular Biotechnology,” Second Edition, by Glick and Pasternak, ASM Press, Washington D.C., 1998, pp. 555-590, the entirety of which is incorporated herein by reference.

For example, a retroviral vector, which is useful when it is desired to have a nucleic acid sequence inserted into the host genome for long term expression, can be packaged in the envelope protein of another virus so that it has the binding specificity and infection spectrum that are determined by the envelope protein (e.g., a pseudotyped virus). In addition, the envelope gene can be genetically engineered to include a DNA element that encodes and amino acid sequence that binds to a cell receptor to create a recombinant retrovirus that infects a specific cell type. Expression of the SERCA2 gene can be further controlled by the use of a cell or tissue-specific promoter. Retroviral vectors have been successfully used to transfect cells with a gene which is expressed and maintained in a variety of ex vivo systems. An adenoviral vector may also be used in the present method. An adenoviral vector infects a wide range of human cells and has been used extensively in live vaccines. Adenoviral vectors used in gene therapy do not integrate into the host genome, and therefore, gene therapy using this system requires periodic administration, although methods have been described which extend the expression time of adenoviral transferred genes, such as administration of antibodies directed against T cell receptors at the site of expression (Sawchuk et al., 1996, Hum. Gene. Ther. 7:499-506). The efficiency of adenovirus-mediated gene delivery can be enhanced by developing a virus that preferentially infects a particular target cell. For example, a gene for the attachment fibers of adenovirus can be engineered to include a DNA element that encodes a protein domain that binds to a cell-specific receptor. Examples of successful in vivo delivery of genes has been demonstrated and are well known. Yet another type of viral vector is based on adeno-associated viruses, which are small, nonpathogenic, single-stranded human viruses. This virus can integrate into a specific site on chromosome 19. This virus can carry a cloned insert of about 4.5 kb, and has typically been successfully used to express proteins in vivo from 70 days to at least 5 months. Demonstrating that the art is quickly advancing in the area of gene therapy, however, a publication by Bennett et al. reported efficient and stable transgene expression by adeno-associated viral vector transfer in vivo for greater than 1 year (Bennett et al., 1999, Proc. Natl. Acad. Sci. USA 96:9920-9925).

Suitable cells to transfect with a recombinant nucleic acid molecule according to the present invention may include any mammalian host cell that can be transfected, that normally or endogenously expresses the SERCA2 protein. Host cells can be either untransfected cells or cells that are already transfected with at least one nucleic acid molecule. Host cells according to the present invention can be any epithelial airway cell capable of producing a SERCA2 protein or in which it is desired to produce the SERCA2. A host cell can also be referred to as a target cell or a targeted cell in vivo, in which a recombinant nucleic acid molecule encoding a SERCA2 protein having the biological activity of the SERCA2 is to be expressed. As used herein, the term “target cell” or “targeted cell” refers to a cell to which a recombinant nucleic acid molecule of the present invention is selectively designed to be delivered. The term target cell does not necessarily restrict the delivery of a recombinant nucleic acid molecule only to the target cell and no other cell, but indicates that the delivery of the recombinant molecule, the expression of the recombinant molecule, or both, are specifically directed to a preselected host cell.

Targeting delivery vehicles, including liposomes, are known in the art. For example, a liposome can be directed to a particular target cell or tissue by using a targeting agent, such as an antibody, soluble receptor or ligand, incorporated with the liposome, to target a particular cell or tissue to which the targeting molecule can bind. Targeting liposomes are described, for example, in Ho et al., 1986, Biochemistry 25: 5500-6; Ho et al., 1987a, J Biol Chem 262: 13979-84; Ho et al., 1987b, J Biol Chem 262: 13973-8; and U.S. Pat. No. 4,957,735 to Huang et al., each of which is incorporated herein by reference in its entirety). Ways in which viral vectors can be modified to deliver a nucleic acid molecule to a target cell have been discussed above. Alternatively, the route of administration, as discussed below, can be used to target a specific cell or tissue. For example, intracoronary administration of an adenoviral vector has been shown to be effective for the delivery of a gene cardiac myocytes (Maurice et al., 1999, J. Clin. Invest. 104:21-29). Intravenous delivery of cholesterol-containing cationic liposomes has been shown to preferentially target pulmonary tissues (Liu et al., Nature Biotechnology 15:167, 1997), and effectively mediate transfer and expression of genes in vivo. Other examples of successful targeted in vivo delivery of nucleic acid molecules are known in the art. Finally, a recombinant nucleic acid molecule can be selectively (i.e., preferentially, substantially exclusively) expressed in a target cell by selecting a transcription control sequence, and preferably, a promoter, which is selectively induced in the target cell and remains substantially inactive in non-target cells.

In one embodiment of the present invention, a recombinant nucleic acid molecule of the present invention is administered to a subject in a liposome delivery vehicle, whereby the nucleic acid sequence encoding the SERCA2 protein enters the host cell (i.e., the target cell) by lipofection. A liposome delivery vehicle contains the recombinant nucleic acid molecule and delivers the molecules to a suitable site in a host recipient. According to the present invention, a liposome delivery vehicle comprises a lipid composition that is capable of delivering a recombinant nucleic acid molecule of the present invention, including both plasmids and viral vectors, to a suitable cell and/or tissue in a subject. A liposome delivery vehicle of the present invention comprises a lipid composition that is capable of fusing with the plasma membrane of the target cell to deliver the recombinant nucleic acid molecule into a cell. A liposome delivery vehicle can also be used to deliver a protein, drug, or other regulatory compound to a subject.

A liposome delivery vehicle of the present invention can be modified to target a particular site in a subject (i.e., a targeting liposome), thereby targeting and making use of a nucleic acid molecule of the present invention at that site. Suitable modifications include manipulating the chemical formula of the lipid portion of the delivery vehicle. Manipulating the chemical formula of the lipid portion of the delivery vehicle can elicit the extracellular or intracellular targeting of the delivery vehicle. For example, a chemical can be added to the lipid formula of a liposome that alters the charge of the lipid bilayer of the liposome so that the liposome fuses with particular cells having particular charge characteristics. Other targeting mechanisms include targeting a site by addition of exogenous targeting molecules (i.e., targeting agents) to a liposome (e.g., antibodies, soluble receptors or ligands).

A liposome delivery vehicle is preferably capable of remaining stable in a subject for a sufficient amount of time to deliver a nucleic acid molecule of the present invention to a preferred site in the subject (i.e., a target cell). A liposome delivery vehicle of the present invention is preferably stable in the subject into which it has been administered for at least about 30 minutes, more preferably for at least about 1 hour and even more preferably for at least about 24 hours. A preferred liposome delivery vehicle of the present invention is from about 0.01 microns to about 1 microns in size.

Suitable liposomes for use with the present invention include any liposome. Preferred liposomes of the present invention include those liposomes commonly used in, for example, gene delivery methods known to those of skill in the art. Preferred liposome delivery vehicles comprise multilamellar vesicle (MLV) lipids and extruded lipids. Methods for preparation of MLV's are well known in the art. According to the present invention, “extruded lipids” are lipids which are prepared similarly to MLV lipids, but which are subsequently extruded through filters of decreasing size. Small unilamellar vesicle (SUV) lipids can also be used in the composition and method of the present invention. In one embodiment, liposome delivery vehicles comprise liposomes having a polycationic lipid composition (i.e., cationic liposomes) and/or liposomes having a cholesterol backbone conjugated to polyethylene glycol. In a preferred embodiment, liposome delivery vehicles useful in the present invention comprise one or more lipids selected from the group of DOTMA, DOTAP, DOTIM, DDAB, and cholesterol.

Preferably, the transfection efficiency of a nucleic acid:liposome complex of the present invention is at least about 1 picogram (pg) of protein expressed per milligram (mg) of total tissue protein per microgram (μg) of nucleic acid delivered. More preferably, the transfection efficiency of a nucleic acid:liposome complex of the present invention is at least about 10 pg of protein expressed per mg of total tissue protein per μg of nucleic acid delivered; and even more preferably, at least about 50 pg of protein expressed per mg of total tissue protein per μg of nucleic acid delivered; and most preferably, at least about 100 pg of protein expressed per mg of total tissue protein per μg of nucleic acid delivered.

Complexing a liposome with a nucleic acid molecule of the present invention can be achieved using methods standard in the art. A suitable concentration of a nucleic acid molecule of the present invention to add to a liposome includes a concentration effective for delivering a sufficient amount of recombinant nucleic acid molecule into a target cell of a subject such that the SERCA2 protein encoded by the nucleic acid molecule can be expressed in a an amount effective to inhibit the growth of the target cell or to inhibit or promote angiogenesis at a tissue site. Preferably, from about 0.1 μg to about 10 μg of nucleic acid molecule of the present invention is combined with about 8 nmol liposomes. In one embodiment, the ratio of nucleic acids to lipids (μg nucleic acid:nmol lipids) in a composition of the present invention is preferably at least from about 1:10 to about 6:1 nucleic acid:lipid by weight (i.e., 1:10=1 μg nucleic acid:10 nmol lipid).

According to the method of the present invention, a host cell is preferably transfected in vivo as a result of administration to a subject of a recombinant nucleic acid molecule, or ex vivo, by removing cells from a subject and transfecting the cells with a recombinant nucleic acid molecule ex vivo.

In some embodiments, the agent may include a SERCA2 activator compound that increases the biological activity of the SERCA2 protein. Such compound may act directly on the endogenous SERCA2 protein to increase or enhance or stimulate its biological activity. Such compounds may be pharmacological activators or stimulators of the SERCA2 protein. Such activator compounds are known in the art and many are commercially available. Examples include, without limitation, PST2744 or Istaroxime ((E,Z)-3-((2-aminoethoxy)imino) androstane-6,17-dione hydrochloride), Memnopeptide A, JTV-519, and CDN1054. Such compounds may further include Beta-adrenergic stimulators, examples of which include, without limitation, albuterol and xopenex. Such compounds may also include growth factor, examples of which include, without limitation, IGF (insulin like growth factor) and EGF (epithelial growth factor). Such compounds may also include a drug, such as rosiglitazone In other embodiments, such agent may act indirectly on the SERCA2 protein to increase or enhance or stimulate its biological activity, and may be an inhibitor of an inhibitor of SERCA2 or an activator of an activator of the SERCA2 protein.

Optionally, the agent may be a protein, nucleic acid molecule, antibody, or a compound that is a product of rational drug design (i.e., drugs) that increases the biological activity of the SECA2 protein.

According to the present invention, the agent that increases the biological activity of the SERCA2 protein, may be administered with a pharmaceutically acceptable carrier, which includes pharmaceutically acceptable excipients and/or delivery vehicles, for delivering the agent to a subject (e.g., a liposome delivery vehicle). As used herein, a pharmaceutically acceptable carrier refers to any substance suitable for delivering a therapeutic composition useful in the method of the present invention to a suitable in vivo or ex vivo site. Preferred pharmaceutically acceptable carriers are capable of maintaining the agent of the present invention in a form that, upon arrival of the agent to a target cell, the agent is capable of entering the cell and increasing the SERCA2 activity in the cell. Suitable excipients of the present invention include excipients or formularies that transport or help transport, but do not specifically target a nucleic acid molecule to a cell (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- or o-cresol, formalin and benzol alcohol. Compositions of the present invention can be sterilized by conventional methods and/or lyophilized.

One type of pharmaceutically acceptable carrier includes a controlled release formulation that is capable of slowly releasing a composition of the present invention into an animal. As used herein, a controlled release formulation comprises the agent that increases the biological activity of the SERCA2 protein in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Natural lipid-containing delivery vehicles include cells and cellular membranes. Artificial lipid-containing delivery vehicles include liposomes and micelles. A delivery vehicle of the present invention can be modified to target to a particular site in a subject, thereby targeting and making use of a nucleic acid molecule at that site. Suitable modifications include manipulating the chemical formula of the lipid portion of the delivery vehicle and/or introducing into the vehicle a targeting agent capable of specifically targeting a delivery vehicle to a preferred site, for example, a preferred cell type.

As discussed above, a composition of the present invention is administered to a subject in a manner effective to deliver the agent to a target cell. When a SERCA2 protein or a homolog thereof is to be delivered to a target cell in a subject, it is administered in a manner whereby the delivered SERCA2 protein is active in the target cell. When a SERCA2 recombinant molecule is to be delivered to a target cell in a subject, it is administered in a manner whereby the SERCA2 protein encoded by the recombinant nucleic acid molecule is expressed in the target cell. When a SERCA2 activator compound is to be delivered to a target cell in a subject, the composition is administered in a manner effective to deliver the SERCA2 regulatory compound to the target cell, whereby the compound can act on the cell so that the expression or biological activity of the SERCA2 is increased. Suitable administration protocols include any in vivo or ex vivo administration protocol.

According to the present invention, an effective administration protocol (i.e., administering a composition in an effective manner) comprises suitable dose parameters and modes of administration that result in increase in the biological activity of the SERCA2 protein, in a target cell of a subject, so that the subject obtains some measurable, observable or perceived benefit from such administration. Effective dose parameters can be determined by experimentation using in vitro cell cultures, in vivo animal models, and eventually, clinical trials if the subject is human. Effective dose parameters can be determined using methods standard in the art for a particular disease or condition that the subject has or is at risk of developing. Such methods include, for example, determination of survival rates, side effects (i.e., toxicity) and progression or regression of disease.

According to the present invention, preferred routes of administration will be apparent to those of skill in the art, depending on the type of delivery vehicle used, whether the compound is a protein, nucleic acid, or other compound (e.g., a drug) and the level of disease or condition experienced by the subject. Preferred methods of in vivo administration include, but are not limited to, intravenous administration, intraperitoneal administration, intramuscular administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracerebral, nasal, oral, pulmonary administration, impregnation of a catheter, and direct injection into a tissue. These administrations can be performed using methods standard in the art. Oral delivery can be performed by complexing a therapeutic composition of the present invention to a carrier capable of withstanding degradation by digestive enzymes in the gut of an animal. Examples of such carriers, include plastic capsules or tablets, such as those known in the art. One method of local administration is by direct injection. Administration of a composition locally within the area of a target cell refers to injecting the composition centimeters and preferably, millimeters from the target cell or tissue.

The agent may be provided in any suitable form, including without limitation, a tablet, a powder, an effervescent tablet, an effervescent powder, a capsule, a liquid, a suspension, a granule or a syrup.

In accordance with the present invention, a suitable single dose of a recombinant nucleic acid molecule encoding a SERCA2 protein as described herein is a dose that is capable of transfecting a host cell and being expressed in the host cell at a level sufficient, in the absence of the addition of any other factors or other manipulation of the host cell, to treat the pulmonary disease when administered one or more times over a suitable time period. Doses can vary depending upon the cell type being targeted, the route of administration, the delivery vehicle used, and the disease or condition being treated.

Another embodiment of the present invention relates to a method (i.e., an assay) for diagnosing a pulmonary disease or the potential therefor in a subject. In one aspect of this embodiment, the method includes the steps of: (a) detecting a level of expression or activity of the SERCA2 protein in a test sample from a subject to be diagnosed; and (b) comparing the level of expression or activity of the SERCA2 in the test sample to a normal level of SERCA2 expression or activity established from a control sample. According to the present invention, detection of the SERCA2 can be achieved by any known method that detects the expression of the SERCA2 or measures the activity of the SERCA2 protein. Detection of a statistically significant difference in SERCA2 expression or activity in the test sample, as compared to the control level of SERCA2 expression or biological activity, is an indicator of the pulmonary disease in the test sample as compared to cells in the control sample.

This method of the present invention has several different uses. First, the method can be used to diagnose the pulmonary disease, or the potential for the pulmonary disease in a subject. The subject can be an individual who is suspected of having the pulmonary disease, or an individual who is presumed to be healthy, but who is undergoing a routine or diagnostic screening for the presence of the pulmonary disease. The terms “diagnose”, “diagnosis”, “diagnosing” and variants thereof refer to the identification of a disease or condition on the basis of its signs and symptoms. As used herein, a “positive diagnosis” indicates that the disease or condition, or a potential for developing the disease or condition, has been identified. In contrast, a “negative diagnosis” indicates that the disease or condition, or a potential for developing the disease or condition, has not been identified. Therefore, in the present invention, a positive diagnosis (i.e., a positive assessment) of the pulmonary disease, such as CF, or the potential therefor, means that the indicators (e.g., signs, symptoms) of the pulmonary disease according to the present invention (i.e., a change in SERCA2 expression or biological activity as compared to a baseline control) have been identified in the sample obtained from the subject. Such a subject can then be prescribed treatment to reduce or eliminate the pulmonary disease. Similarly, a negative diagnosis (i.e., a negative assessment) for the pulmonary disease or a potential therefor means that the indicators of the pulmonary disease or a likelihood of developing the pulmonary disease as described herein (i.e., a change in SERCA2 expression or biological activity as compared to a baseline control) have not been identified in the sample obtained from the subject. In this instance, the subject is typically not prescribed any treatment, but may be reevaluated at one or more timepoints in the future to again assess presence of the pulmonary disease. Baseline levels for this particular embodiment of the method of diagnosis of the present invention are typically based on a “normal” or “healthy” sample from the same bodily source as the test sample (i.e., the same tissue, cells or bodily fluid), as discussed in detail below.

The first step of the method of the present invention includes detecting SERCA2 expression or biological activity in a test sample from a subject. According to the present invention, the term “test sample” can be used generally to refer to a sample of any type which contains cells to be evaluated by the present method, including but not limited to, a sample of isolated cells, a tissue sample and/or a bodily fluid sample. Bodily fluids suitable for sampling include, but are not limited to, bronchoalveolar lavage (BAL) fluid, mucus, blood, seminal fluid, saliva, breast milk, bile and urine.

According to the present invention, a sample of isolated cells is a specimen of cells, typically in suspension or separated from connective tissue which may have connected the cells within a tissue in vivo, which have been collected from an organ, tissue or fluid by any suitable method which results in the collection of a suitable number of cells for evaluation by the method of the present invention. The cells in the cell sample are not necessarily of the same type, although purification methods can be used to enrich for the type of cells that are preferably evaluated. Cells can be obtained, for example, by scraping of a tissue, processing of a tissue sample to release individual cells, or isolation from a bodily fluid. A tissue sample, although similar to a sample of isolated cells, is defined herein as a section of an organ or tissue of the body which typically includes several cell types and/or cytoskeletal structure which holds the cells together. One of skill in the art will appreciate that the term “tissue sample” may be used, in some instances, interchangeably with a “cell sample”, although it is preferably used to designate a more complex structure than a cell sample.

Once a sample is obtained from the subject, the sample is evaluated for detection of SERCA2 expression or biological activity in the cells of the sample. The phrase “SERCA2 expression” can generally refer to SERCA2 mRNA transcription or SERCA2 protein translation. Preferably, the method of detecting SERCA2 expression or biological activity in the subject is the same or qualitatively equivalent to the method used for detection of SERCA2 expression or biological activity in the sample used to establish the baseline level.

Methods suitable for detecting SERCA2 transcription include any suitable method for detecting and/or measuring mRNA levels from a cell or cell extract. Such methods include, but are not limited to: polymerase chain reaction (PCR), reverse transcriptase PCR (RT-PCR), in situ hybridization, Northern blot, sequence analysis, gene microarray analysis (gene chip analysis) and detection of a reporter gene. Such methods for detection of transcription levels are well known in the art, and many of such methods are described in detail in the attached examples, in Sambrook et al., Molecular Cloning: A Laboratory Manual, Cold Spring Harbor Labs Press, 1989 and/or in Glick et al., Molecular Biotechnology Principles and Applications of Recombinant DNA, ASM Press, 1998; Sambrook et al., ibid., and Glick et al., ibid. are incorporated by reference herein in their entireties.

SERCA2 expression can also be identified by detection of SERCA2 translation (i.e., detection of SERCA2 protein in a sample). Methods suitable for the detection of SERCA2 protein include any suitable method for detecting and/or measuring proteins from a cell or cell extract. Such methods include, but are not limited to, immunoblot (e.g., Western blot), enzyme-linked immunosorbant assay (ELISA), radioimmunoassay (RIA), immunoprecipitation, immunohistochemistry and immunofluorescence. Particularly preferred methods for detection of proteins include any single-cell assay, including immunohistochemistry and immunofluorescence assays. Such methods are well known in the art. Furthermore, antibodies against certain of the SERCA2 described herein are known in the art and are described in the public literature, and methods for production of antibodies that can be developed against SERCA2 are well known in the art. The activity of the SERCA2 protein can be measured by any method known in the art or as described herein.

The method of the present invention includes a step of comparing the level of SERCA2 expression or biological activity detected in step (a) to a baseline level (also known as a control level) of SERCA2 expression or biological activity established from a control sample. According to the present invention, a “baseline level” is a control level, and in some embodiments (but not all embodiments, depending on the method), a normal level, of SERCA2 expression or activity against which a test level of SERCA2 expression or biological activity (i.e., in the test sample) can be compared. Therefore, it can be determined, based on the control or baseline level of SERCA2 expression or biological activity, whether a sample to be evaluated for a pulmonary disease has a measurable increase, decrease, or substantially no change in SERCA2 expression or biological activity, as compared to the baseline level. The term “negative control” or “normal control” used in reference to a baseline level of SERCA2 expression or biological activity typically refers to a baseline level established in a sample from the subject or from a population of individuals which is believed to be normal (i.e., non-disease). In another embodiment, a baseline can be indicative of a positive diagnosis of the disease. Such a baseline level, also referred to herein as a “positive control” baseline, refers to a level of SERCA2 expression or biological activity established in a cell sample from the subject, another subject, or a population of individuals, wherein the sample was believed to be diseased.

In yet another embodiment, the baseline level can be established from a previous sample from the subject being tested, so that the disease of a subject can be monitored over time and/or so that the efficacy of a given therapeutic protocol can be evaluated over time. In such embodiments, the baseline level of SERCA2 expression or biological activity is determined from at least one measurement of SERCA2 expression or biological activity in a previous sample from the same subject. Such a sample is from the subject at a different time point than the sample to be tested. In one embodiment, the previous sample resulted in a negative diagnosis (i.e., no disease, or potential therefor, was identified). In this embodiment, a new sample is evaluated periodically (e.g., at annual physicals), and as long as the subject is determined to be negative for the disease, an average or other suitable statistically appropriate baseline of the previous samples can be used as a “negative control” for subsequent evaluations. For the first evaluation, an alternate control can be used, as described below, or additional testing may be performed to confirm an initial negative diagnosis, if desired, and the value for SERCA2 expression or biological activity can be used thereafter. This type of baseline control is frequently used in other clinical diagnosis procedures where a “normal” level may differ from subject to subject and/or where obtaining an autologous control sample at the time of diagnosis is not possible, not practical or not beneficial.

In another embodiment, the previous sample from the subject may have resulted in a positive diagnosis (i.e., the disease was positively identified). In this embodiment, the baseline provided by the previous sample is effectively a positive control for the disease, and the subsequent samplings of the subject are compared to this baseline to monitor the progress of the disease and/or to evaluate the efficacy of a treatment that is being prescribed for the disease. In this embodiment, it may also be beneficial to have a negative baseline level of SERCA2 expression or biological activity (i.e., a normal cell baseline control), so that a baseline for regression of the disease can be set. Monitoring of a subject's disease can be used by the clinician to modify the disease treatment for the subject based on whether an increase or decrease in SERCA2 is indicated.

Another method for establishing a baseline level of SERCA2 expression or biological activity is to establish a baseline level of SERCA2 expression or biological activity from control samples, and preferably control samples that were obtained from a population of matched individuals. It is preferred that the control samples are of the same sample type as the sample type to be evaluated for SERCA2 expression or biological activity (e.g., the same cell type, and preferably from the same tissue or organ). According to the present invention, the phrase “matched individuals” refers to a matching of the control individuals on the basis of one or more characteristics which are suitable for the disease to be evaluated. For example, control individuals can be matched with the subject to be evaluated on the basis of gender, age, race, or any relevant biological or sociological factor that may affect the baseline of the control individuals and the subject (e.g., preexisting conditions, consumption of particular substances, levels of other biological or physiological factors). To establish a control or baseline level of SERCA2 expression or biological activity, samples from a number of matched individuals are obtained and evaluated for SERCA2 expression or biological activity. The sample type is preferably of the same sample type and obtained from the same organ, tissue or bodily fluid as the sample type to be evaluated in the test subject. The number of matched individuals from whom control samples must be obtained to establish a suitable control level (e.g., a population) can be determined by those of skill in the art, but should be statistically appropriate to establish a suitable baseline for comparison with the subject to be evaluated (i.e., the test subject). The values obtained from the control samples are statistically processed using any suitable method of statistical analysis to establish a suitable baseline level using methods standard in the art for establishing such values.

It will be appreciated by those of skill in the art that a baseline need not be established for each assay as the assay is performed but rather, a baseline can be established by referring to a form of stored information regarding a previously determined baseline level of SERCA2 expression for a given control sample, such as a baseline level established by any of the above-described methods. Such a form of stored information can include, for example, but is not limited to, a reference chart, listing or electronic file of population or individual data regarding “normal” (negative control) or disease positive SERCA2 expression; a medical chart for the subject recording data from previous evaluations; or any other source of data regarding baseline SERCA2 expression that is useful for the subject to be diagnosed.

After the level of SERCA2 expression or biological activity is detected in the sample to be evaluated for the pulmonary disease, such level is compared to the established baseline level of SERCA2 expression or biological activity, determined as described above. Also, as mentioned above, preferably, the method of detecting used for the sample to be evaluated is the same or qualitatively and/or quantitatively equivalent to the method of detecting used to establish the baseline level, such that the levels of the test sample and the baseline can be directly compared. In comparing the test sample to the baseline control, it is determined whether the test sample has a measurable decrease or increase in SERCA2 expression or biological activity over the baseline level, or whether there is no statistically significant difference between the test and baseline levels. After comparing the levels of SERCA2 expression or biological activity in the samples, the final step of making a diagnosis, or monitoring of the subject can be performed as discussed above.

In order to establish a positive diagnosis, the level of SERCA2 activity is modulated as compared to the established baseline by an amount that is statistically significant (i.e., with at least a 95% confidence level, or p<0.05). Preferably, detection of at least about a 10% change in SERCA2 expression or biological activity in the sample as compared to the baseline level results in a positive diagnosis of the disease for said sample, as compared to the baseline. More preferably, detection of at least about a 15%, at least about 20%, or at least about 25% change in SERCA2 expression or biological activity in the sample as compared to the baseline level results in a positive diagnosis of CF for said sample, as compared to the baseline. More preferably, detection of at least about a 50% change, and more preferably at least about a 70% change, and more preferably at least about a 90% change, or any percentage change greater than 10% in 1% increments (i.e., 5%, 6%, 7%, 8% . . . ) in SERCA2 expression or biological activity in the sample as compared to the baseline level results in a positive diagnosis of CF for said sample.

Once a positive diagnosis is made using the present method, the diagnosis can be substantiated, if desired, using any suitable alternate method of detection of the disease.

Included in the present invention are kits for assessing a pulmonary disease in a subject. The assay kit includes: (a) reagents for detecting SERCA2 expression or activity in a test sample (e.g., a probe that hybridizes under stringent hybridization conditions to a nucleic acid molecule encoding the SERCA2 or a fragment thereof; RT-PCR primers for amplification of mRNA encoding the SERCA2 or a fragment thereof; and/or an antibody, antigen-binding fragment thereof or other antigen-binding peptide that selectively binds to the SERCA2); and (b) reagents for detecting a control marker characteristic of a cell type in the test sample (e.g., a probe that hybridizes under stringent hybridization conditions to a nucleic acid molecule encoding a protein marker; PCR primers which amplify such a nucleic acid molecule; and/or an antibody, antigen binding fragment thereof, or antigen binding peptide that selectively binds to the control marker in the sample).

The reagents for detecting of part (a) and or part (b) of the assay kit of the present invention can be conjugated to a detectable tag or detectable label. Such a tag can be any suitable tag which allows for detection of the reagents of part (a) or (b) and includes, but is not limited to, any composition or label detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., 3H, 125I, 35S, 14C, or 32P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

In addition, the reagents for detecting of part (a) and or part (b) of the assay kit of the present invention can be immobilized on a substrate. Such a substrate can include any suitable substrate for immobilization of a detection reagent such as would be used in any of the previously described methods of detection. Briefly, a substrate suitable for immobilization of a means for detecting includes any solid support, such as any solid organic, biopolymer or inorganic support that can form a bond with the means for detecting without significantly effecting the activity and/or ability of the detection means to detect the desired target molecule. Exemplary organic solid supports include polymers such as polystyrene, nylon, phenol-formaldehyde resins, acrylic copolymers (e.g., polyacrylamide), stabilized intact whole cells, and stabilized crude whole cell/membrane homogenates. Exemplary biopolymer supports include cellulose, polydextrans (e.g., Sephadex®), agarose, collagen and chitin. Exemplary inorganic supports include glass beads (porous and nonporous), stainless steel, metal oxides (e.g., porous ceramics such as ZrO2, TiO2, Al2O3, and NiO) and sand.

Another embodiment of the present invention may include a method to evaluate the efficacy of a treatment of a pulmonary disease in a subject. In this method, the levels of expression of SERCA2 may be determined in a sample taken from the subject before and after administering the treatment, and the before and after levels of SERCA2 expression may be compared. The level of SERCA2 expression after administering the treatment may be greater than before administering the treatment, less than before administering the treatment, or may remain about the same as before administering the treatment. Depending on the results of the comparison of the SERCA2 expression levels before and after administering the treatment, the treatment plan may be revised to provide better therapeutic outcome. The level of SERCA2 expression after administering the treatment may be monitored over a period of time. The monitoring may continue even after the initial treatment plan has ended to detect whether the disease has returned. The step of detecting may comprise detecting SERCA2 mRNA in the test sample, or detecting SERCA2 protein in the test sample, or detecting SERCA2 protein biological activity in the test sample. Preferably, the method of detecting the level of SERCA2 expression before and after administering the treatment is the same. In a preferred embodiment, the pulmonary disease is Cystic Fibrosis.

The following examples are provided for the purpose of illustration and are not intended to limit the scope of the present invention. Any variations which occur to the skilled artisan are intended to fall within the scope of the present invention. Each publication, sequence or other reference disclosed below and elsewhere herein is incorporated herein by reference in its entirety, to the extent that there is no inconsistency with the present disclosure.

### EXAMPLES

The Following materials and methods are applicable to all Examples.

Cell culture: Cell lines used herein and cell culturing conditions applicable to all examples are as follows. Primary human bronchial epithelial cells (HBEs) were obtained from non-CF and CF lungs under protocol and consent form approved by the University of North Carolina School of Medicine Committee on the Protection of the Rights of Human Subjects. Epithelial cells were removed from the lower trachea and bronchi by protease XIV digestion and cells were plated in BEGM medium on collagen-coated dishes as described previously (23). Approximately, 5×10⁵ passage 2 cells were seeded onto mm diameter type VI collagen (Sigma) coated Millicell CM inserts (0.4 μM pore size, Millipore Corporation, Bedford, Mass.) and following confluence on day 4-5 were maintained at an air liquid interface (ALI). Additional primary human bronchial epithelial cells were isolated in the Carl White laboratory from donor airway tissues obtained from National Disease Research Interchange, NDRI with approval of National Jewish Institutional Committee for the Protection of the Rights of Human Subjects (NJIRB). The airway epithelial cell lines used were IB3-1 and a “corrected” cystic fibrosis (CF) cell line that was derived from IB3-1 cells stably transfected with wild-type CFTR (C-38) (ATCC, Manassas, Va.). IB3-1 and C-38 cells were grown in LHC-8 media (Invitrogen, Carlsbad, Calif.) supplemented with 10% FBS and penicillin/streptomycin. CFBE4lo- (CF4lo-) and CFBE45o- (CF45o-) and a wild-type airway epithelial cell line, 16HBE14o- (16HBEo-), were provided by Prof. D. Gruenert (California Pacific Medical Center Research Institute, University of California at San Francisco). These cell lines were cultured in Eagle's minimal essential medium (Invitrogen, Carlsbad, Calif.) supplemented with 10% fetal bovine serum, L-glutamine, and penicillin/streptomycin at 37° C. under 5% CO₂. 16HBEo- cells with stable expression of sense (16HBE-S) and antisense CFTR (16HBE-AS) oligonucleotides were cultured as described previously (24). Additionally, minimally transformed primary bronchial epithelial UNCN3T from the lab of Dr. Randell was also used (25). Additional non-CF and CF cell line pair used was Calu-3 and JME CF/15 (89). All experiments comparing primary non-CF and CF cells were performed with polarized cultures grown simultaneously and matched for passage number, the number of cells plated, and days in culture.

Protein concentration: Protein concentration in cell lysates was determined using the BioRad DC protein assay kit (Bio-Rad, Hercules, Calif.) in a 96-well plate with bovine serum albumin as a standard.

Statistical methods: All statistical calculations were performed with JMP and SAS software (SAS Institute, Cary, N.C.). Means were compared either by two-tailed t test for comparison between two groups or one-way analysis of variance (ANOVA) followed by the Tukey-Kramer test for multiple comparisons for analyses involving three or more groups. A P value of <0.05 was considered significant. For analysis of data with distribution that was not normal, Welch's test was used to determine significance.

### Example 1

This example illustrates that the expression of SERCA2 protein in decreased in CF cell lines.

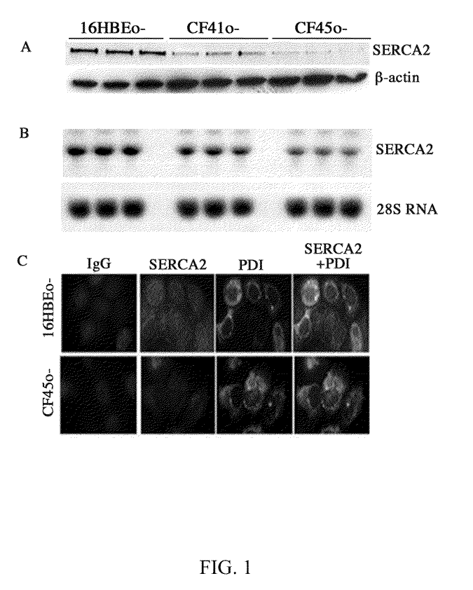

Lysates from cultures of 16HBEo-, CF4lo- and CF45o- cells were analyzed for SERCA2 protein and RNA expression using Western and Northern blot as indicated below. FIGS. 1A, and 1B are representative Western (experiments repeated 6 times) and Northern blots (experiments repeated 3 times). To localize SERCA2, cells grown on glass coverslips were fixed and co-immunostained for SERCA2 (red) and endoplasmic reticulum (ER) specific protein, protein disulphide isomerase (PDI, green) (1C). The FIG. 1 represents one data set from an experiment performed in duplicate. The individual experiment was repeated 3 times. The experiments are described in detail below.

SERCA2 protein expression was determined by Western blot in non-CF16HBEo-) and CF (CF4lo- and CF35o-) cell lysates. Western blots were performed as previously described in detail (64) and the membranes were probed with rabbit polyclonal antibodies against SERCA2 (Affinity Bioreagents, Golden, Colo.) at 1:1,000 dilution, overnight at 4° C. Blots were then washed again with TBS-T and incubated with mouse anti-rabbit peroxidase-conjugated IgG (Bio-Rad, Hercules, Calif.) at 1:2,000 dilution, for 1 h at room temperature. Immunoreactive bands were detected using an ECL detection kit (Pierce, Rockford, Ill.) followed by exposure to Hyperfilm (Amersham Pharmacia Biotech Inc. UK).

Expression of SERCA2 protein was decreased in both CF cell lines (CF4lo- and CF45o-) as compared to the non-CF 16HBEo- cells (FIG. 1A). Quantitation of SERCA2/β-actin intensity yielded 3.51±0.27, 1.76±0.23, and 2.59±0.29 arbitrary units for 16HBEo-, CF4lo-, and CF45o- cells, respectively. Similarly a statistically significant decrease in SERCA2 protein was observed in IB3-1 cells versus C-38 cells, (0.22±0.02 vs. 1.24±0.01 SERCA2/β-actin intensity units, respectively) and in JME/CF15 versus Calu-3 cells (0.38±0.01 vs. 0.62±0.01 SERCA2/β-actin intensity units, respectively).

SERCA2 mRNA expression was determined by Northern blot in non-CF 16HBEo-) and CF (CF4lo- and CF35o-) cell lysates. The cells were washed twice with Hank's balanced salt solution (HBSS) and harvested in guanidine isothiocyanate solution (66). Total cell RNA was then purified with CsCl centrifugation. Equal amounts of RNA (15 μg) were resolved on a 1% agarose-2.5 M formamide gel in a 20 mM MOPS buffer, pH 7.4, containing 1 mM EDTA. A standard Northern blot procedure (66) was used to transfer the RNA to a nylon membrane (Micro Separations, Westborough, Mass.). The SERCA2 cDNAs was obtained from Origene, Rockville, Md. The cDNAs were labeled with a randomly primed labeling kit (Invitrogen, Carlsbad, Calif.) and [³²P] dCTP (ICN, Irvine, Calif.). Blots were hybridized with the probe and autoradiographed. Quantitative analysis of Northern blots was performed with ImageQuant 1.11 (Molecular Dynamics, Sunnyvale, Calif.) after the blots were exposed to a phosphor screen. Northern blot data were normalized for loading efficiency with a random prime-labeled 28S rRNA probe (Ambion, Austin, Tex.). Decreased SERCA2 mRNA was observed (FIG. 1B) in CF4lo- and CF45o- cells relative to that found in non-CF 16HBEo- cells.

Experiments were also performed to immunolocalize SERCA2. Cells grown on glass coverslips in 6-well plates were fixed in 4% paraformaldehyde (PFA) for 10 minutes, rinsed in TBS and permeabilized with 0.4% Triton-X-100 in 10 mM sodium citrate for 20 minutes. After blocking in 5% donkey serum for 20 minutes, cells were incubated with rabbit anti-SERCA2 (Affinity Bioreagents, Golden, Colo.) and mouse anti-PDI (Gene Tex Inc, San Antonio, Tex.) for 1 hour. Negative controls included normal rabbit or mouse IgG at the same concentration as the primary antibodies used. Secondary antibodies, Texas red-conjugated donkey anti-rabbit or FITC-conjugated donkey anti-mouse were then applied for 60 minutes. Cells were mounted on slides with Prolong Gold-DAPI and allowed to dry overnight. Slides were viewed using a Zeiss, Axiovert 200M fluorescent microscope and digital images recorded using Slidebook software (both from Intelligent Imaging Innovations, Denver, Colo.).

The results again indicated diminished SERCA2 expression in CF cells relative to non-CF cells (FIG. 1C). In non-CF cells, SERCA2 was located in ER, as demonstrated by the enhanced ‘merged’ staining for the ER marker protein disulfide isomerase (PDI) and SERCA2 in these cells, whereas CF cells demonstrated decreased overall SERCA2 staining intensity, preservation of ER staining for PDI, and diminished ‘merged’ signal for PDI and SERCA2 co-staining

### Example 2

This Example illustrates that the decreased expression of SERCA2 in CF cells was not an effect due to diminished ER mass in these cells, and that decreased SERCA2 expression resulted in a substantial overall decrease of SERCA activity in CF cells.

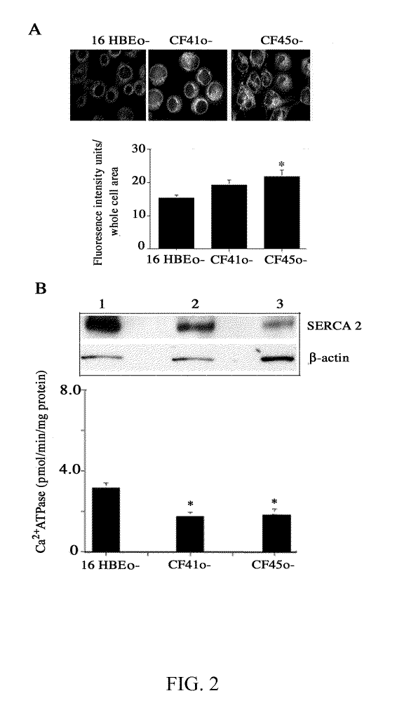

To determine that the decreased expression of SERCA2 in CF cells was not an effect due to diminished ER mass in these cells, estimation of endoplasmic reticulum (ER) content was performed. Live CF and non-CF cells cultured in chambered coverglass were stained using an ER-specific fluorescent dye, ER-Tracker Blue-White DPX (Molecular Probes) to quantify the ER density (FIG. 2A, upper panel). FIG. 2A, lower panel shows the quantification of fluorescence intensity per whole cells area. Similar to what was seen with PDI staining, CF cell cultures showed similar or greater ER staining intensity with the fluorescent dye. Specifically, CF45o- cells had significantly greater staining intensity than 16HBEo- cells, and CF4lo- cells showed a similar, nonsignificant trend (FIG. 2A, lower panel). These findings are consistent with previous reports of increased apical ER density in CF cells (29B).

Next, SERCA2 protein expression and activity was measured in purified microsomal membranes. The method used for isolation of the microsomes is one modified from that described before (67). Cells were washed with 5 ml of prewarmed (37° C.) phosphate-buffered saline solution. The cells were then scraped in a solution of cold (prechilled on ice) phosphate-buffered saline with 5 mM EDTA before being transferred and collected in a single tube on ice. The cells were pelleted by centrifugation at 4000×g for 15 min at 4° C. The supernatant was discarded, and the resulting pellet was resuspended gently with 10 ml of phosphate-buffered saline prior to centrifugation, as before. The supernatant was then discarded, and the cells were resuspended gently with 5 ml of prechilled (on ice) hypotonic solution (10 mM Tris, pH 7.5, 0.5 mM MgCl₂). The resuspended cells were then incubated on ice for 10 min prior to the addition of 0.1 mM phenylmethylsulfonyl fluoride and 4 μg/ml leupeptin. The lysed cells were homogenized using a glass Dounce homogenizer for 30 strokes. The homogenate was then diluted with an equal volume of buffer (0.5 M sucrose, 6 mM 2-mercaptoethanol, 40 μM CaCl₂, 300 mM KCl, and 20 mM Tris, pH 7.5) before being centrifuged at 1000×g for 10 min at 4° C. The supernatant was removed and made up to 0.6 M with KCl by the addition of an appropriate volume of a 2.5 M solution, prior to centrifugation at 100,000×g for 60 min at 4° C. to obtain the microsomal membrane fraction. The microsomal pellet was then resuspended in buffer (0.25 M sucrose, 0.15 M KCl, 3 mM 2-mercaptoethanol, 20 μM CaCl₂, and 10 mM Tris, pH 7.5). The microsomal membranes were rehomogenized before being aliquoted and snap-frozen with liquid nitrogen, prior to storage at −80° C.

Microsomal extracts (20 μg) were loaded on 7.5% polyacrylamide gel, and Western blot analysis was performed for SERCA2 expression, as described in Example 1. In microsomal membrane (ER) preparations, CF cell lines CF4lo- and CF45o- had decreased SERCA2 protein expression, both in absolute terms and per β-actin protein expressed, relative to non-CF cell line 16HBEo- (FIG. 2B, upper panel).

The Ca²⁺-dependent ATPase activity of microsomal membranes was measured using the phosphate liberation assay (68). Briefly, microsomal extracts (typically 20 μg) were resuspended in 200 μl of buffer (45 mM Hepes/KOH (pH 7.0), 6 mM MgCl₂, 2 mM NaN₃, 0.25 mM sucrose), supplemented with 5 μg/ml A23187 ionophore and EGTA and CaCl₂ to give a free [Ca²⁺] of 2 μM. Assays were preincubated at 37° C. for 10 min prior to the addition of ATP with a final concentration of 6 mM to initiate activity. The reactions were then incubated at 37° C. for 40 min, before the addition of 50 μl of 6.5% trichloroacetic acid, and the reactions were then stored on ice for 10 min before centrifugation for 5 min at 20,000×g. Supernatant (100 μl) was added to 150 μl of buffer (11.25% (v/v) acetic acid, 0.25% (w/v) copper sulfate, and 0.2 M sodium acetate, pH 4.0). Ammonium molybdate solution (5% w/v, 25 μl) was then added, followed by the addition of 25 μl of ELAN reagent (2% w/v p-methyl-aminophenol sulfate and 5% w/v sodium sulfite). The samples were mixed, and the blue color was allowed to develop for 10 min prior to measuring the absorption at 870 nm using a Dynatech Laboratories enzyme-linked immunosorbent assay (ELISA) plate reader. The amount of inorganic phosphate liberated was determined by comparison with known phosphate standards. The activities were also determined in the absence of the addition of Ca²⁺ to determine non-Ca²⁺ dependent ATPase activity. All activities were calculated as pmol/min/mg of microsomal protein. Stock solutions of thapsigargin, were prepared in dimethyl sulphoxide (DMSO), when added to the assays, the amount of DMSO never exceeded 1% v/v, which was shown not to have any effect on the Ca²⁺-dependent ATPase activity of the microsomal membranes.

In these microsomal membrane preparations, total thapsigargin-inhibitable Ca²⁺ ATPase (SERCA) activity was decreased by approximately 50% in the two CF cell lines relative to the non-CF cells (FIG. 2B, lower panel). Because thapsigargin is a specific inhibitor of SERCA, these findings indicate that diminished SERCA2 expression resulted in a substantial overall decrease of SERCA activity in CF cells.

FIG. 2 summarizes the results described herein. FIG. 2A shows staining of cells for ER and the quantification of fluorescence intensity per whole cells area. For each of 3 cell lines about 20 cells were analyzed. Results show means of data and * indicates significant difference (p<0.05) from non-CF 16HBEo- cells (n=3). FIG. 2B shows Western blot for SERCA2 expression, and a bar chart showing activity in purified microsomal membranes. * indicates significant difference from 16HBEo- cells (p<0.05) (n=3, represents 3 individual experiments).

### Example 3

This Example illustrates that SERCA2 expression is increased in air-liquid interface (ALI) cultures of primary CF airway epithelial cells.

SERCA2 expression was estimated in air-liquid interface (ALI) cultures of differentiated primary non-CF (14 donors) and CF (8 donors) cells using immunohistochemistry and Western blot analysis.

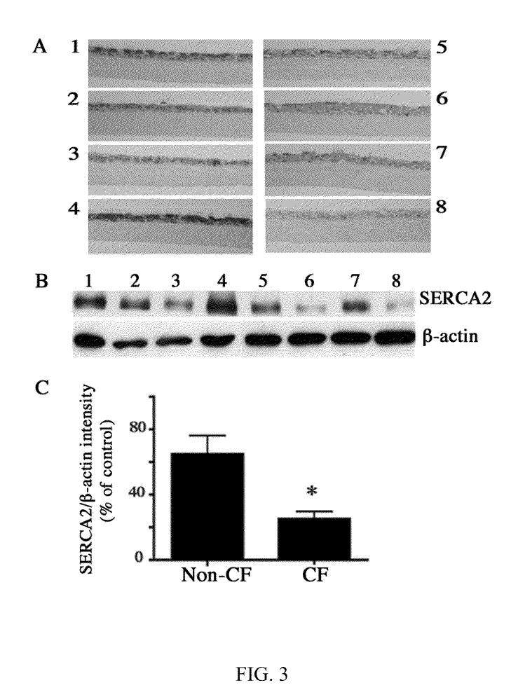

Using either technique, expression of SERCA2 in epithelial cells varied from donor to donor (demographics provided in Table 1 below), despite comparable passage number and days in culture (FIGS. 3A and 3B).

TABLE 1
Demographics of airway tissue donors for cells

| | | Age/ | | |
|---|---|---|---|---|
| Donor | | Sex | | |
| No. | Category | (yr) | COD | Genotype |
| 1 | NTD | 44/M | IVH | |
| 2 | NTD | 48/M | Head trauma | |
| 3 | NTD | 16/F | Head trauma | |
| 4 | (Tissue from | 11/F | | |
| | UNC) | | | |
| 5 | NTD | 40/M | CVA | |
| 6 | (Tissue from | 22/M | | |
| | Miami) | | | |
| 7 | NTD | 47/M | Anoxia | |
| 8 | NTD | 24/M | MVA/head | |
| | | | trauma | |
| 9 | NTD | 68/F | Head trauma | |
| 10 | TD | 14/M | | |
| 11 | NTD | 22/M | CVA | |
| 12 | TD | 42/M | antifreeze | |
| | | | poisoning | |
| 13 | (Tissue from | 61/M | | |
| | UNC) | | | |
| 14 | CF transplant | 40/M | | ΔF508/? |
| 15 | CF transplant | 22/F | | Not Genotyped |
| 16 | CF transplant | 14/F | | ΔF508/ΔF508 |
| 17 | CF transplant | 35/F | | ΔF508/ΔF508 |
| 18 | CF transplant | 24/M | | ΔF508/ΔF508 |
| 19 | CF transplant | 34/M | | ΔF508/ΔF508 |
| 20 | CF transplant | 31/M | | ΔF508/1898 + IG > A |
| 21 | CF transplant | 17/M | | ΔF508/ΔF508 |

Definition of abbreviations:
COD = cause of death,
NTD = nontransplant donor, lung unsuitable because of acute injury, age, etc.;
TD = excess airway from lung transplant donor;
? = mutation not identified,
IVH = intraventricular hemorrhage,
CVA = cardiovascular arrest,
MVA = motor vehicle accident.

However, mean SERCA2 protein expression (Western blot) was decreased by 67% in airway epithelial cells from CF donors, relative to non-CF (FIG. 3C). SERCA2 expression was also analyzed at 7 days of culture, a stage at which cells are undifferentiated, and also at 30 days of culture, at which time cells are more differentiated (30), in primary non-CF and CF airway epithelial cells from 4 different donors, to determine if extent of differentiation could affect SERCA2 expression. CF airway epithelial cells had decreased SERCA2 expression at 7 days as well as at 30 days of culture as compared to non-CF (data not shown). Thus, SERCA2 protein expression in primary polarized CF airway epithelial cells differed from the non-CF cells to a similar extent as seen in CF versus non-CF cell lines.

FIG. 3 summarizes the results discussed here regardinh the SERCA2 expression in air-liquid interface (ALI) cultures of primary non-CF and CF airway epithelial cells. FIG. 3A shows representative images of in situ immunohistochemistry for SERCA2 expression in cultures fixed with 4% PFA and stained with 3,3-diaminobenzidine and H₂O₂ for detection of HRP-coupled mouse secondary antibody (data shown is from cells from 4 individual (1-4) non-CF donors and 4 individual (5-8) CF donors (one of three separate experiments)). Identical staining conditions were used for staining non-CF and CF sections. For Western blot, lysates from the ALI cultures of the cells from above donors were analyzed by SDS/PAGE on 4-15% gradient gels, and the proteins were transferred electrophoretically onto nitrocellulose membranes. The membranes were probed with the SERCA2 antibodies at a 1/1,000 dilution (FIG. 3B). FIG. 3C shows quantification of Western blots for SERCA2 expression in ALI cultures of cells from non-CF and CF donors (14 non-CF and 8 CF) analysed in 3 separate experiments (The mean of the non-CF group is the control value); >60% of CF donors were F508 double mutant and ˜90% had F508 mutation for one allele in the CFTR gene). The bars represent means of data and * indicates significant difference (p<0.05) from non-CF cells (results of 3 individual experiments).

### Example 4

This example illustrates that SERCA2 expression is decreased in proximal and distal airways from human CF and non-CF lungs.

Immunohistochemistry was used to estimate SERCA2 expression in lung tissue from CF and non-CF individuals. Non-CF and CF lung tissues were obtained from National Disease Research Interchange, NDRI with approval of National Jewish Institutional Committee for the Protection of the Rights of Human Subjects (NJIRB). Paraffin-embedded sections (5 μm) were dewaxed, rehydrated, and exposed to antigen retrieval (vegetable steamer for 25 min followed by a 20 min cool down). After quenching of endogenous peroxidase and alkaline phosphatase for 10 min. (Dual Blocker; Dako, Carpinteria, Calif.), the nonspecific binding was blocked (Serum Free Protein Block; Open Biosystems, Huntsville, Ala.). The sections were then incubated for 60 min on a Dako autostainer with anti-human antibodies against SERCA2 (mouse monoclonal; 1:400, Affinity Bioreagents, Golden, Colo.), with protein concentration-matched mouse IgG (BD Pharmingen, San Diego, Calif.) for negative controls. After incubation with labeled polymer-HRP-antimouse (horseradish peroxidase-labeled polymer conjugated to goat antimouse immunoglobulin) (EnVision +HRP, Dako) for 30 min, color was developed by 3,3-diaminobenzidine (BioCare Medical, Walnut Creek, Calif.) combined with H₂O₂. Counterstaining was performed with hematoxylin (Open Biosystems, Huntsville, Ala.). Identical staining conditions were maintained during staining of non-CF and CF sections. Similarly, for in situ SERCA2 staining in ALI cultures, the cells growing on inserts were fixed with 4% PFA. After paraffin embedding SERCA2 or hematooxylin and eosin staining was then performed on the deparaffinized slides with sections of each insert on edge. The methods were developed in collaboration with and stains performed by IHCtech (Aurora, Colo.).

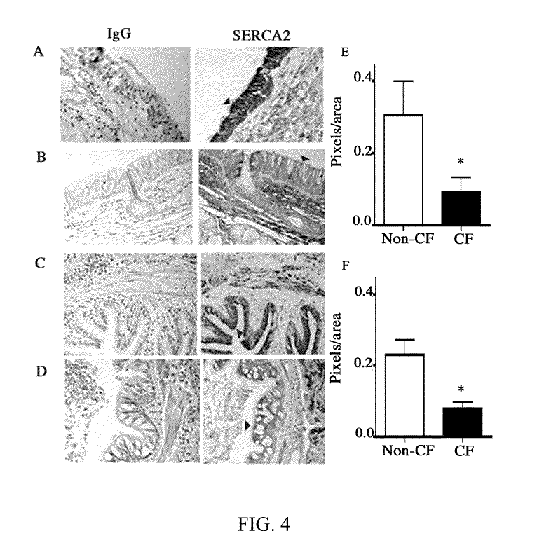

SERCA2 staining was predominantly localized to airway epithelium and to smooth muscle cells in airway and arterial/arteriolar walls. SERCA2 staining was decreased in both bronchial and bronchiolar epithelium of CF relative to non-CF patients (FIG. 4A-D). CF airway epithelium also differed from non-CF in that the extent of mucus cell hyperplasia was greater in the former than latter.

Quantitation of SERCA2 staining (excluding the mucus-containing regions) revealed a significant decrease in both bronchial and bronchiolar epithelium of CF relative to non-CF patients (FIGS. 4E & 4F). The demographics of the CF and non-CF individuals are provided in Table 2.

TABLE 2
Demographics of airway tissue donors for IHC

| | | Age/Sex | |
|---|---|---|---|
| Donor No. | Category/COD | (yr) | Genotype |
| 1. | NAT/Cancer | 56/M | |
| 2. | MVA | 42/M | |
| 3. | NAT/Cancer | 63/M | |
| 4. | MVA | 36/F | |
| 5. | CVA | 44/F | |
| 6. | Cerebral edema | 12/M | ΔF508/ΔF508 |
| 7. | ARF | 14/F | ΔF508/? |
| 8. | CF | 19/F | ΔF508/? |
| 9. | CF | 18/M | ΔF508/ΔF508 |
| 10. | CF | 41/M | ΔF508/? |

Definition of abbreviations:
COD = cause of death,
NAT = normal adjacent tissue,
? = mutation not identified,
ARF = Acute Respiratory Failure,
MVA = motor vehicle accident,
CVA = cardio vascular arrest.

FIG. 4 shows the results described in this example. In FIG. 4A Left panel is the non-specific IgG control, and right panel has arrowheads ( [symbol] ) indicating SERCA2 staining in epithelium. SERCA2 staining was found predominantly in the epithelium of non-CF bronchi (n=5 donors) (A) and bronchioles (C), and it was significantly less intense in the epithelium of CF airways (n=5 donors) (B & D). Graph E on the right shows the quantitation of SERCA2 staining (SERCA2-IgG) in the non-CF and CF bronchi. The regions containing mucus were excluded during quantitation. For each tissue, two SERCA2 and two IgG stained sections were analyzed and 10 non-mucus areas per section were randomly selected for quantitation using Image-Pro Plus version 4.0 (Media Cybernetics, Silver Spring, Md.). Similarly SERCA2 staining in the non-CF and CF bronchioles was quantified (graph F).

### Example 5

This example illustrates that SERCA3 expression is increased in CF airway epithelial cells and cell lines.

SERCA3 is the only other known SERCA isoform expressed in lung. It differs from SERCA2 in that it has a low affinity for calcium. SERCA3 mRNA and protein expression was determined by Northern blot and Western blot as previously described in detail in Example 1, using SERCA3 cDNA from Origene, Rockville, Md. and anti-SERCA3 rabbit polyclonal antibodies from Affinity Bioreagents Golden, Colo., respectively.

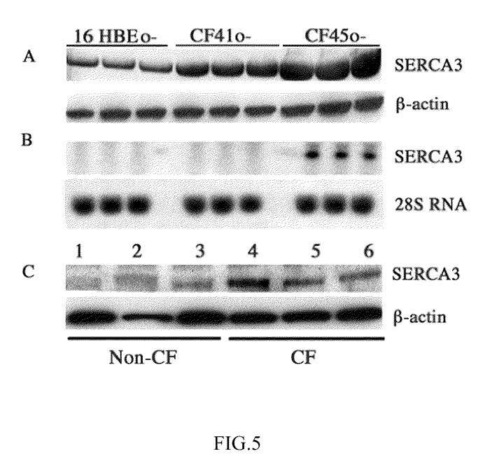

These results are shown in FIG. 5. FIG. 5A, top row, represents the Western blot for SERCA3 from whole cell lysates (20 μg of protein/lane) from non-CF and CF bronchial airway epithelial cell lines. Membranes were also probed for β-actin to verify equal loading of protein. FIG. 5B represents the Northern blot for SERCA3 mRNA expression. About 15 μg of RNA was subjected to Northern blot analysis, and SERCA3 was identified using ³²P-labeled cDNA probe. Before hybridization membranes were analyzed with UV light exposure for visualization of 18S and 28S RNA bands to verify equality of RNA loading and transfer. Equal loading was further established by 28S RNA (bottom panel) analysis. Cell lysates from ALI cultures of primary airway epithelial cells from 3 non-CF (1-3) and 3 CF (4-6) subjects were also assessed for SERCA3 expression by Western blot (FIG. 5C). Results represent 3 individual experiments.

By contrast to SERCA2, SERCA3 protein expression was increased in both CF cell lines, CF4lo- and CF45o-, relative to that seen in the non-CF cell line 16HBEo- (FIG. 5A). Steady-state SERCA3 mRNA expression was not consistently elevated in CF cell lines, being elevated in CF45o- but not CF4lo- relative to 16HBEo- (FIG. 5B). However, SERCA3 protein expression was increased in primary polarized airway epithelial cells from CF versus non-CF donors (FIG. 5C). These findings suggest a compensatory response of SERCA3 expression to the downregulation of SERCA2 in CF.

### Example 6

This example illustrates that SERCA2 protein expression is decreased by pharmacologic inhibition of CFTR function.

To investigate the role of CFTR function in mediating SERCA2 expression, the specific CFTR inhibitor CFTRᵢₙₕ172 was used. Primary bronchial epithelial cells were cultured for short-term duration on collagen coated inserts and then treated with 20 μM CFTRᵢₙₕ172 for 24 h. Cell lysates were prepared, and Western blot analysis was performed as described in Example 1 (20 μg protein loaded).

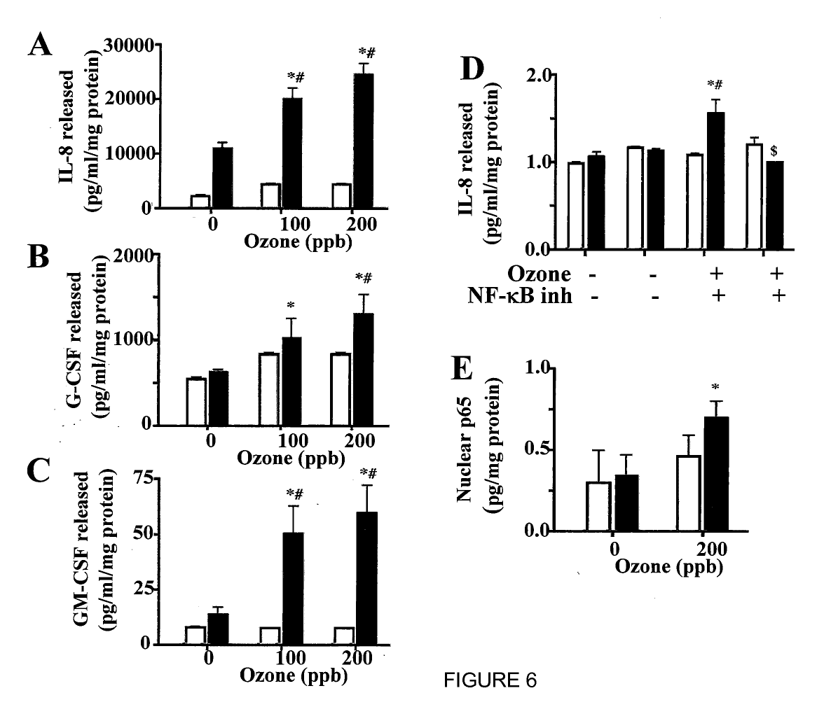

Results are shown in FIG. 6. FIG. 6A shows SERCA2 expression in CFTRᵢₙₕ172-treated primary bronchial epithelial cells (Lane 1-3 are untreated control & 4-6 are CFTRᵢₙₕ172-treated cells). FIG. 6B shows the quantitative data for SERCA2 expression with and without CFTRᵢₙₕ172 treatment. The bars represent means of data and * indicates significant difference (p<0.05) from non-CF cells (results of 3 individual experiments are shown). FIG. 6C is a representative blot showing effect of CFTRᵢₙₕ172 on SERCA2 expression treatment in CF IB3-1 cells and CFTR corrected C-38 cells. Panel D shows the quantitative data. The bars represent mean of data and * indicates significant difference (p<0.05) from untreated cells n=3 (experiment repeated twice).

Relatively short-term (24 h) incubation of primary human bronchial epithelial (HBE; air-liquid interface; FIG. 6A) and the wt-CFTR-corrected C-38 (FIG. 6C) cells, respectively, with CFTRᵢₙₕ172 (20 μM) caused a significant decrease in SERCA2 protein levels. By contrast, incubation of the parent CF cell line IB3-1 with the same concentration of the inhibitor did not cause a further decrease in its expression of SERCA2 (FIG. 6C). Exposure of these cells to the inhibitor for this duration did not cause cytotoxicity.

### Example 7

This example illustrates that SERCA2 protein expression is decreased by genetic inhibition of CFTR function.

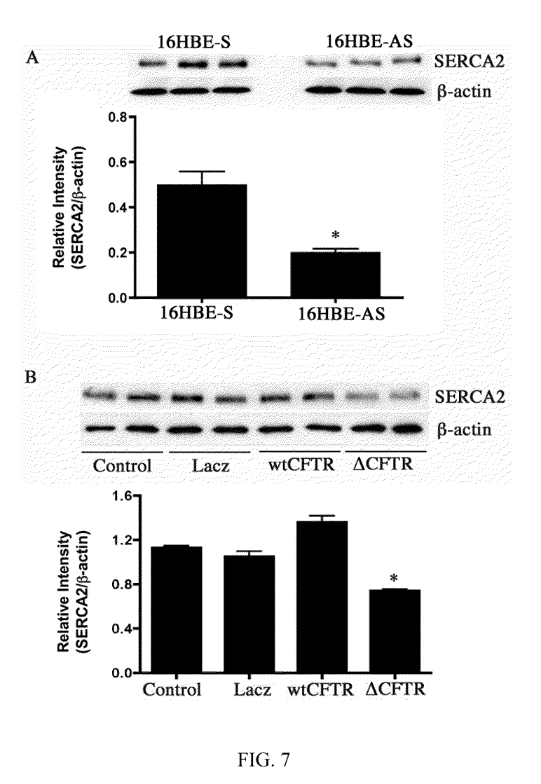

The effect of interference with CFTR function was evaluated by specific genetic approaches by either inhibiting functional CFTR expression by antisense CFTR oligonucleotides (FIG. 7A) or by overexpressing mutated CFTR (FIG. 7B).

### SERCA2 Protein Expression is Decreased by Genetic Inhibition of CFTR Function

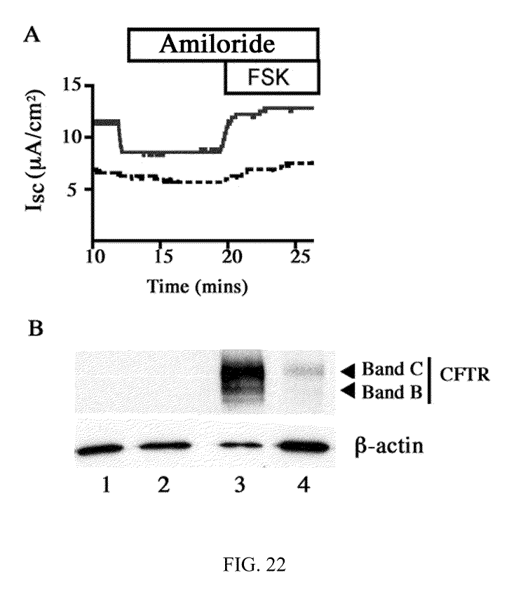

Polarized cultures of 16HBEo- cell line were stably transfected with sense (S) and antisense (AS) CFTR oligonucleotide. The cAMP-dependent chloride conductance was measured in these cells as follows. 16HBE-S and 16HBE-AS were seeded on snapwell permeable supports (Corning Costar) at a density of 5×10⁵ cells/cm². At confluence (˜14 day of ALI culture), the inserts were mounted in Ussing chambers (WPI, Sarasota, Fla.) filled on the basolateral side with 10 ml of Krebs bicarbonate solution containing (in mM): 120 NaCl, 3.3 KH₂PO₄, 0.8 K₂HPO₄, 1.2 MgCl₂, 1.2 CaCl₂, 25 NaHCO₃, 10 glucose. On the apical side, 10 mM mannitol was added instead of glucose to avoid activation of the apical electrogenic Na⁺-glucose cotransporter. During the experiment, this solution was gassed with 95% O₂/5% CO₂. Experiments were conducted at 37° C. The short-circuit current (Isc) was monitored continuously using a DVC1000 voltage clamp (WPI, Sarasota, Fla.) and the PD was measured every 5-10 min. Cell preparations were allowed to equilibrate until stabilization of bioelectric variables took place, which required ˜20-30 min. Basal bioelectric activity was monitored for 10 min before addition of drugs. Pharmacologic agents were added to the apical bathing solutions and bioelectric activity was monitored for 5-15 min thereafter. Amiloride (10 μM) and forskolin (10 μM), were added sequentially. The cAMP-dependent chloride conductance was absent in antisense CFTR oligonucleotides containing cells (16HBE-AS) (FIG. 22A).

Cell lysates were prepared from polarized cultures of 16HBEo- cell line stably transfected with sense (S) and antisense (AS) CFTR oligonucleotide and analyzed for SERCA2 expression by Western blot, as described in Example 1. FIG. 7A shows the effect of inhibiting functional CFTR expression by antisense CFTR oligonucleotides on SERCA2 expression. The bars represent means of data and * indicates significant difference (p<0.05) from control (16HBE-S) cells. The experiment was repeated more than 3 times.

Notably, SERCA2 protein, corrected for β-actin expression, was diminished in CFTR antisense-expressing cells by about two-thirds relative to sense-expressing cells (FIG. 7A), comparable to the extent of reduction of SERCA2 expression in primary CF relative to non-CF airway cells.

### SERCA2 Protein Expression is Decreased by Expression of Mutant ΔF508 CFTR

The ΔF508 CFTR mutation causes diminished channel activity, impaired processing, and reduced CFTR protein stability at the cell surface. In addition, this mutation inhibits gating of CFTR channels, resulting in a diminished rate of opening (31B). Because this is the most common CFTR mutation, the effect of its overexpression in airway epithelial cells on SERCA2 expression was measured.

Studies were conducted in minimally transformed primary human bronchial epithelial cells (UNCN3T) transiently transduced using adenoviral vectors. Transduction of wild-type CFTR, mutated CFTR (ΔF508) and GFP-encoding adenoviral vectors was carried out as described before (26). The vectors (H5′0.040CMVGFP-CFTR and H5′0.040CMVGFP-ΔF508) were provided by Vector Core at the University of Pennsylvania, PA, as described elsewhere (27). The recombinant viruses were added to the cell cultures (Multiplicity of infection, MOI 10:1) on day 3 for 17 hours. The transduction efficiency was estimated by observing green fluorescence of adenoviral GFP-transduced cells. For detection of CFTR, anti-CFTR antibody 570 (dilution 1:500) was obtained from University of North Carolina (UNC) CFTR Antibody Distribution Program sponsored by Cystic Fibrosis Foundation Therapeutics (CFFT).

FIG. 7B shows the effect of inhibiting functional CFTR expression by overexpressing mutated CFTR (B) on SERCA2 expression. The lower panel of FIG. 7B represents results of SERCA2 expression analysis in lysates obtained from control and adenovirally-transduced minimally transformed primary human bronchial epithelial cells (UNCN3T) grown on collagen coated culture dishes. The bars represent means of data and * indicates significant difference (p<0.05) from control (LacZ) cells. The experiment repeated three times (n=3 per condition).

Relative to nontransduced, LacZ-overexpressing, and wt-CFTR overexpressing cells (increased CFTR expression by Ad.wt-CFTR is shown by Western blot in FIG. 22B), ΔF508 CFTR-overexpressing cells had decreased SERCA2 protein expression (FIG. 7B). Taken together, the results from use of these strategies for CFTR functional inhibition demonstrate a link between diminished CFTR function and SERCA2 expression.

### Example 8

This example illustrates the Bcl-2-induced displacement of SERCA2 from caveolae related domains (CRDs) in CF cells.

For the isolation of CRDs, the microsomal membranes were lysed in 2.0 ml of ice-cold 0.5 M sodium carbonate buffer (pH 11.0) using 20 strokes of a Dounce glass homogenizer followed by sonication and CRDs were prepared as described before (69,70). To determine the distribution of CRD-associated proteins within the gradient, each fraction was analyzed by SDS-PAGE on 4-20% gradient gel, followed by Western blot analysis with appropriate antibodies.

Interaction of Bcl-2 with SERCA2 has been described previously (20, 32). Bcl-2 binds to and inactivates SERCA. In addition, it causes SERCA displacement from the CRDs of the ER membrane (19, 20). Sucrose density gradient fractionation of purified microsomes from non-CF 16HBEo- cells and CF45o- cells was performed to localize SERCA2 in the CRDs.

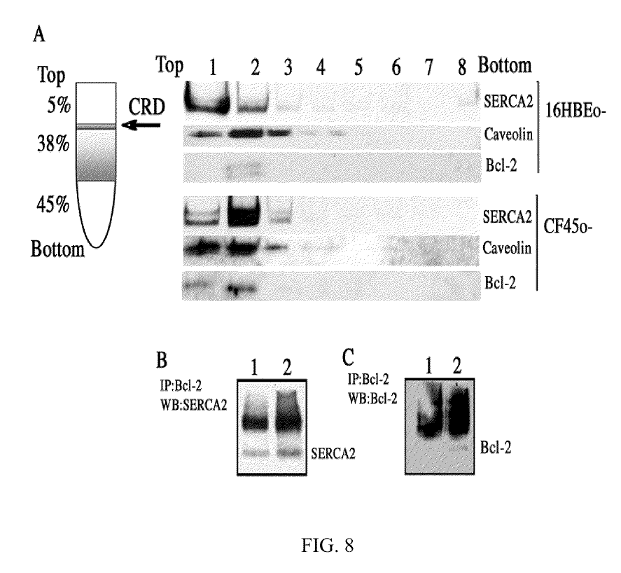

Purified microsomes from 16HBEo- and CF45o- cells were lysed in ice-cold 0.5 M sodium carbonate buffer. The homogenate was adjusted to 45% (w/v) sucrose by the addition of 90% sucrose in the MBS buffer and placed in the bottom of an ultracentrifuge tube. A discontinuous sucrose gradient was established by overlaying this solution with 4 ml of 38% sucrose and 3 ml of 5% sucrose. The tubes were then centrifuged at 4° C. for 16-18 h at 130,000×g and fractions were manually collected from the top of the gradient. To determine the distribution of CRD-associated proteins within the gradient, each fraction was analyzed by SDS-PAGE, followed by Western blot analysis with SERCA2 and caveolin antibody (FIG. 8A). The Western blot shown is representative of findings in three separate sucrose gradient centrifugation/Western blot experiments. Four fractions were collected from the top of the gradient, where fraction 1 represents the initial CRD fraction. SERCA2 of 16HBEo- cells primarily localized in the CRD fraction whereas the major SERCA2 band of CF45o- cells migrated deeper into the gradient. Thus, sucrose density gradient centrifugation of microsomes from CF preparations showed that SERCA2 was predominantly recovered in fraction 2, whereas non-CF samples showed SERCA2 predominantly in fraction 1.

FIGS. 8B & 8C show Western blot of SERCA2 and Bcl-2 using immunoprecipitate of microsomal fractions from (1) 16HBEo- and (2) CF45o- using Bcl-2 antibody. Mouse monoclonal antibodies against Bcl-2 was obtained from BD Biosciences, San Jose, Calif. Bcl-2 immunoprecipitation experiments were carried out as described previously (65). SERCA2 co-immunoprecipitated with Bcl-2 in both 16HBEo- and CF45o- cells (FIG. 8B). However, an increased amount of Bcl-2 was detected in CRDs and the immunoprecipitates from CF cell microsomes (FIG. 8C). These figures indicate the presence of increased Bcl-2 expression and Bcl-2-SERCA2 complexes in CF. The increase in Bcl-2-SERCA2 complexes in CF cells occurred despite the fact that SERCA2 protein was decreased by about 50% in the CF cell line. These findings indicate a mechanism for decline in ER-associated SERCA2 expression and total SERCA activity due to Bcl-2 association.

### Example 9

This example illustrates that Bcl-2 expression is increased in cellular compartments of CF cells and that diminished CFTR expression plays a role in increased Bcl-2 expression.

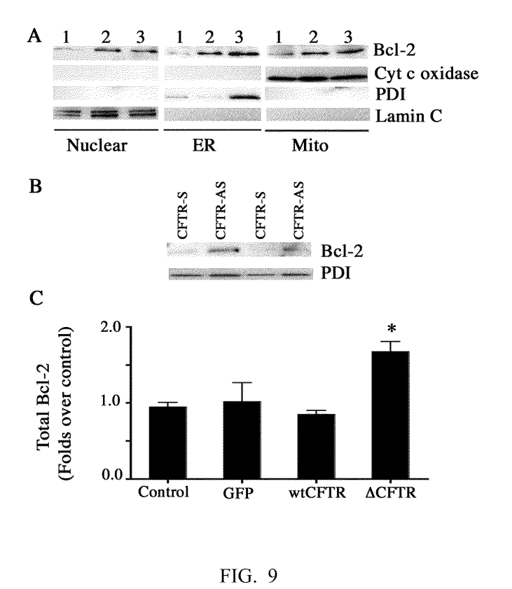

FIG. 9A shows the Western blot analysis of Bcl-2 distribution in the nucleus, ER and mitochondria of 16HBEo-, CF4lo- and CF45o- cells. Nuclear, ER and mitochondrial fractions were prepared from (1) 16HBEo-, (2) CF4lo- and (3) CF45o- cells. Western blots were performed using antibodies against Bcl-2, cytochrome c oxidase (mitochondrial marker), protein disulphide isomerase (PDI, ER specific protein) and lamin C (nuclear marker). Cytochrome c oxidase, Lamin C and p65 antibodies (Affinity Bioreagents, Golden, Colo.) were used at a dilution of 1:1000.

Increased Bcl-2 protein was observed in each of these cellular compartments of the two CF, relative to the non-CF, cell lines. Analysis of total Bcl-2 content in the ER membrane (M) of 16HBEo- and CF45o- cells using ELISA (Table 3) confirmed findings obtained by Western blot. Bcl-2 contents of whole cell lysates (WCL) from differentiated primary human non-CF and CF bronchial epithelial cells showed a pattern of increase in CF cells, but the difference was not significantly different (Table 3).

TABLE 3
Total Bcl-2 expression in non-CF and CF bronchial epithelial cells

| | Total Bcl-2 |
|---|---|
| Cells | (ng/mg protein) |
| Differentiated primary non-CF bronchial epithelial | 7.74 ± 1.41 |
| cells (WCL) (n = 5 donors) | |
| Differentiated primary non-CF bronchial epithelial | 10.55 ± 1.50 |
| cells (WCL) (n = 4 donors) | |
| 16HBEo-(M) | 8.90 ± 0.11 |
| CF45o-(M) | 14.50 ± 0.12* |

WCL; whole cell lysate
M; microsomal membrane preparations
*indicates significant difference from non-CF 16HBEo-p < 0.05.

To determine if decreased CFTR function plays a role in the enhanced expression of Bcl-2 ER microsomes were isolated from 16HBEo- cell line stably transfected with sense (S) and antisense (AS) CFTR oligonucleotide. Increased expression of Bcl-2 was observed in cells in which CFTR was decreased by expression of antisense CFTR oligonucleotides (FIG. 9B). This indicates that deficient CFTR expression is sufficient to increase Bcl-2 expression.

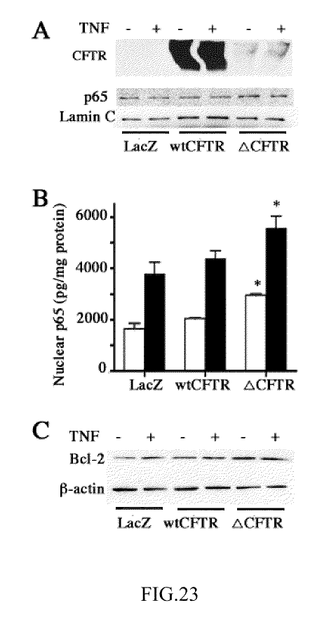

Bcl-2 analysis was also performed in cellular lysates obtained from control and adenovirally-transduced primary human bronchial epithelial cells grown on collagen-coated culture dishes using ELISA. FIG. 9C shows the total Bcl-2 content in these lysates. The bars represent means of data of two individual experiments (n=4) and * indicates significant difference (p<0.05) from control. Cells expressing ΔF508 CFTR showed a ˜2-fold increase in Bcl-2 expression as compared to the controls (FIG. 9C). Direct and/or indirect effects of altered CFTR function may lead to persistent endogenous activation of NF-κB in CF airway epithelial cells (33, 34). NF-κB regulates the transcription of Bcl-2 (35, 36). Determination was made of Bcl-2 content and NF-κB activation (by measuring nuclear p65) of control and TNF-treated primary HBE cells that were adenovirally transduced with ΔF508 CFTR (FIG. 23).

Primary airway epithelial cells cultured on collagen coated dishes were adenovirally transduced with LacZ, wtCFTR and ΔCFTR. After 24 h cells were treated with TNF (10 ng/ml, 18 h). Cell lysates and nuclear lysates were prepared. Results are shown in FIG. 23. Top panel of 23A shows CFTR expression. Lower panel shows Western blot of p65 in the nuclear lysate. Panel B is quantification of nuclear translocation of NF-κB as measured by ELISA. The bars represent mean of data and * indicates significant difference (p<0.05) from untreated cells. The experiment was repeated two times with n=3. FIG. 23C is a Western blot for Bcl-2 in whole cell lysates. The experiment was repeated 3 times and representative blot is shown.

There was no change in CFTR expression with and without TNF treatment in the lacZ, wtCFTR and ΔF508 CFTR expressing cells. The nuclear lysates, when assessed by Western blot, showed increased p65 in the ΔF508 CFTR expressing cells in both conditions of ΔF508 CFTR, either with or without TNF treatment. Quantitative estimation of p65 in the nuclear lysates using ELISA revealed statistically significant increased nuclear p65 upon TNF treatment in the adenoviral lacZ and wtCFTR transduced cells (FIG. 23B). The ΔF508 CFTR expressing cells had increased basal nuclear p65 that was further enhanced upon TNF treatment. The cells that had increased nuclear p65 had increased Bcl-2 expression (FIG. 23C).

### Example 10

This example illustrates that SERCA2 is required for cell survival in oxidative stress.

Cystic fibrosis airway epithelial cells are continuously exposed to oxidative stress presented directly or indirectly by air pollutants, bacterial endotoxins, pro-inflammatory cytokines and neutrophils. For this reason, the potential impact of diminished SERCA2 expression on airway epithelial cell survival was determined during challenge by three relevant pro-oxidant stimuli: ozone, hydrogen peroxide and TNFα.

SERCA2 siRNA was used to investigate the effect of SERCA2 expression on ozone-induced cell death in primary airway epithelial cells cultured on collagen coated E-well plates. Predesigned human ‘SMARTPOOL’ SERCA2 siRNAs were purchased from Dharmacon (Dharmacon, Lafayette, Colo.). Primary airway epithelial cells not at ALI, were transfected with 50 nM siRNA using DharmaFECT2 siRNA transfection reagent (Dharmacon, Lafayette, Colo.) according to the manufacturer's instructions. Silencer Negative Control siRNA was used as a non-specific siRNA and mock transfection was used as a negative control. Transfection of siRNA in primary human airway epithelial cells has previously been described (28).

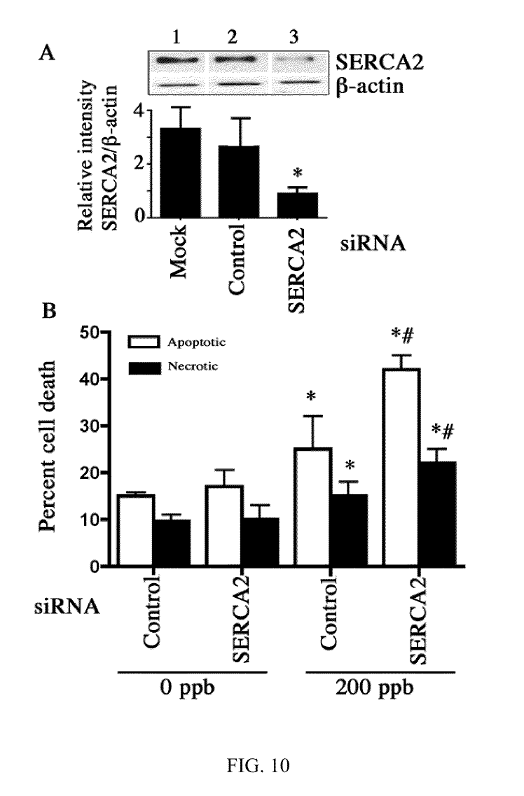

First, it was verified that SERCA2 protein levels were reduced in the cells expressing SERCA2 siRNA. FIG. 10A shows SERCA2 knockdown using SERCA2 siRNA. The top panel is the representative Western blot of SERCA2 expression in primary human bronchial epithelial cells (grown on collagen-coated culture dishes) either (1) mock-transfected or transfected with (2) control or (3) SERCA2 siRNA. The lower panel shows the quantitation of SERCA2 knockdown. The bars represent means of data and * indicates significant difference (p<0.05) from control siRNA-transfected cells (n=3), and represents 3 individual experiments. Transfection with nonspecific siRNA had no effect on SERCA2 protein expression whereas SERCA2 siRNA-treated cells had SERCA2 protein expression decreased by 67% relative to nonspecific siRNA-treated controls. Bcl-2 expression was not affected by SERCA2 knockdown in these cells (data not shown).

For assessing ozone toxicity, primary human airway epithelial cells were transfected either with a control or SERCA2 siRNA for 24 h and exposed to 0 ppb or 200 ppb ozone for 18 h. Following exposure, cells were analyzed for cell death using Vybrant apoptosis assay kit (Molecular Probes, Eugene, Oreg.), as described before (63). In this method, apoptotic cells bearing phosphatidylserine in the plasma membrane outer leaflet were identified as those binding Alexa Fluor 488-labeled annexin V and necrotic cells as those binding fluorescent DNA-binding dye SYTOX Green. The Alexa Fluor-488 labeled annexin-positive apoptotic and Sytox green-positive dead cells were quantified by flow cytometry. Results are shown in FIG. 10B. The columns represent means of data and * indicates significant difference (p<0.05) from 0 ppb controls cells. # indicates significant difference from 200 ppb controls (n=3). This experiment was repeated 4 times.

Increased apoptotic as well as necrotic cell death was observed in the control siRNA-transfected cells upon exposure to 200 ppb ozone for 18 h (FIG. 10B). SERCA2 siRNA increased both apoptotic and necrotic cell death induced by ozone (25.0±7.0% vs. 42.0±3.0% apoptotic cell death in control siRNA vs. SERCA2 siRNA expressing cells).

Besides ozone as an environmental factor toxicity of TNFα and H₂O₂ was also assessed, as these are abundant in CF lungs. With 10 ng/ml TNFα, a concentration close to those found in CF airways (37), there was enhanced cell death in SERCA2 siRNA expressing cells, but the extent of death was minimal (4.17±0.30% in control vs 7.15±0.50% total cell death in SERCA2 siRNA expressing cells). However, when TNFα was combined with IL-1β, another cytokine abundant in CF airways, there was increased apoptotic cell death in control, and 1.7-fold still greater apoptosis in SERCA2 siRNA treated cells (Table 4). The extent of necrosis was not different in the two groups. Similarly, treatment with H₂O₂ caused enhanced apoptotic as well as necrotic cell death in SERCA2 siRNA expressing cells when compared to those expressing control siRNA (Table 4).

TABLE 4
SERCA2 knockdown enhances cell death

| | Control siRNA | | SERCA2 siRNA | |
|---|---|---|---|---|
| Treatment | % Apoptosis | % Necrosis | % Apoptosis | % Necrosis |
| TNF + | 17.53 ± 1.83 | 8.54 ± 0.55 | 29.46 ± 0.72* | 9.53 ± 0.39 |
| IL1β, | | | | |
| 10 ng/ml, | | | | |
| 18 h | | | | |
| H₂O₂, | 9.66 ± 0.33 | 14.9 ± 0.85 | 14.00 ± 1.00* | 28.33 ± 1.66* |
| 500 μM, | | | | |
| 18 h | | | | |

*indicates significant difference from control siRNA treated cells p < 0.05, n = 3 experiment repeated 3 times.

### Example 11

This example illustrates that CFTR inhibition decreases ATP release and cell survival in ozone.

Release of extracellular ATP upon exposure to oxidant gases is critical for airway epithelial cell survival (78,81). This polarized (apical) ATP release is primarily vescicular and regulated by calcium and PI3K dependent pathways. CFTR may also modulate release of ATP (92). Therefore, primary human airway epithelial cells were exposed to 200 ppb ozone with or without CFTR inhibitor 172 (CFTRᵢₙₕ172).

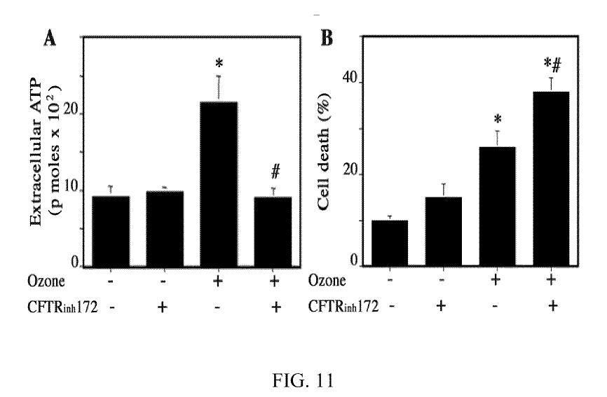

Exposure of airway epithelial cells to ozone at precise levels was carried out in a computer controlled in vitro exposure chamber as described previously (78). Primary human bronchial epithelial cells cultured on collagen-coated 6-well plates were treated with 20 μM CFTRᵢₙₕ172 for 30 min and then exposed to either 0 ppb (−) or 200 ppb (+) ozone. ATP content of the extracellular media was analyzed after 30 min and cell death was estimated after 8 h of ozone exposure as described in the Methods. Results are shown in FIG. 11. The data shown is a representative of 3 experiments (n=6). The bars represent means (SEM) of data and * indicates significant difference (p<0.05) from 0 ppb (− ozone) controls and # indicates significant difference (p<0.05) from 200 ppb exposed cells without CFTRᵢₙₕ172.

Short term (30 min) exposure to ozone of airway epithelial cells caused enhanced accumulation of ATP in the extracellular media (FIG. 11A). Ozone-mediated release of ATP was completely abolished by incubating cells with CFTRᵢₙₕ172. Prolonged exposure to ozone caused enhanced cell death in airway epithelial cells. Treatment with CFTR inhibitor further enhanced the cell death (FIG. 11B). CFTR inhibitor itself had negligible cytotoxicity at the dose and duration of exposure.

### Example 12

This example illustrates that CF cells have decreased ATP release in ozone.

To further understand the role of CFTR and to study cellular responses of cystic fibrosis airway epithelial cells non-CF (16HBEo-) and CF (CF4lo- and CF45o-) cells cultured at air liquid interface were exposed to ozone for various time intervals.

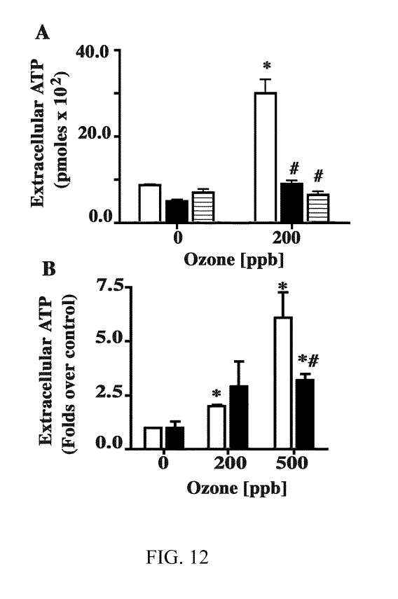

Non-CF 16HBE and CF, CF4lo- and CF45o- were cultured on (30 mm) fibronectin coated inserts. Media was changed before exposure and 200 μl media was added on the apical surface. Apical media was collected at the end of exposure (30 min) and analyzed for ATP content as described in the text. The results are shown in FIG. 12. The data shown in FIG. 12A is a representative experiment performed four times (n=6). The bars represent means of data and * indicates significant difference (p<0.05) from 0 ppb controls and # indicates significant difference (p<0.05) from 200 ppb exposed non-CF cells.

Non-CF 16HBEo- cells (open bars) released ATP that was maximum at 30 min of ozone exposure (FIG. 12A). CF4lo- cells (closed bars) released ATP in the extracellular medium however it was to a lesser extent than the non-CF cells. CF45o- cells (hatched bars) had similar level of ATP in both 0 ppb as well as 200 ppb. Other non-CF and CF airway epithelial cell line pairs viz. C38 and IB-3, and calu-3 and JME CF/15 were used. The CF cells had consistently decreased ATP release upon ozone exposure as compared to the non-CF cells (data not shown). Cells that were stably transfected with sense (non-CF, S-1) and antisense (CF, AS-3) CFTR oligonucleotide were also used. The antisense CFTR expressing cells having biologically inhibited CFTR activity had decreased ATP release in ozone as compared to the controls (S-1 cells).

The ozone-mediated ATP release response of primary airway epithelial cells isolated from non-CF and CF donor proximal airway tissue and cultured at ALI were also compared. The demographics of the donors is provided in Table 5.

TABLE 5
Demographics of airway tissue donors for cells

| | | Age/Sex | | |
|---|---|---|---|---|
| Donor No. | Category | (yr) | COD | Genotype |
| 1 | NTD | 16/F | Head trauma | |
| 2 | (Tissue from | 11/F | | |
| | UNC) | | | |
| 3 | NTD | 40/M | CVA | |
| 4 | (Tissue from | 22/M | | |
| | Miami) | | | |
| 5 | NTD | 24/M | MVA/head | |
| | | | trauma | |
| 6 | TD | 14/M | | |
| 7 | CF transplant | 40/M | | ΔF508/? |
| 8 | CF transplant | 22/F | | ΔF508/ΔF508 |
| 9 | CF transplant | 14/F | | ΔF508/ΔF508 |
| 10 | CF transplant | 35/F | | ΔF508/ΔF508 |
| 11 | CF transplant | 24/M | | ΔF508/ΔF508 |
| 12 | CF transplant | 34/M | | ΔF508/ΔF508 |

FIG. 12B shows quantification of apical ATP release in differentiated ALI cultures of primary cells from non-CF and CF donors (3 non-CF and 3 CF) analyzed in 3 separate experiments (The mean of the non-CF group is the control value). The bars represent means of data and * indicates significant difference (p<0.05) from 0 ppb control and # indicates significant difference (p<0.05) from ozone exposed non-CF cells. Ozone exposure of polarized and differentiated primary airway epithelial cells caused enhanced ATP release in non-CF cells in a dose dependent manner at 200 and 500 ppb ozone (FIG. 12B). ATP concentration of apical media of CF cells was not significantly different from non-CF cells at 200 ppb ozone. However, ATP release due to higher ozone concentrations (500 ppb) was significantly greater in non-CF cells than CF cells.

### Example 13

This example illustrates the increased ozone-induced toxicity in CF airway cells.

A systemic investigation of ozone (at close to ambient concentrations) toxicity using non-CF (16HBEo-) and CF (CF4lo- and CF45o-) cells cultured at ALI was performed. Several components of cellular toxicity individually to assess the effect of ozone exposure were examined.

Membrane Damage:

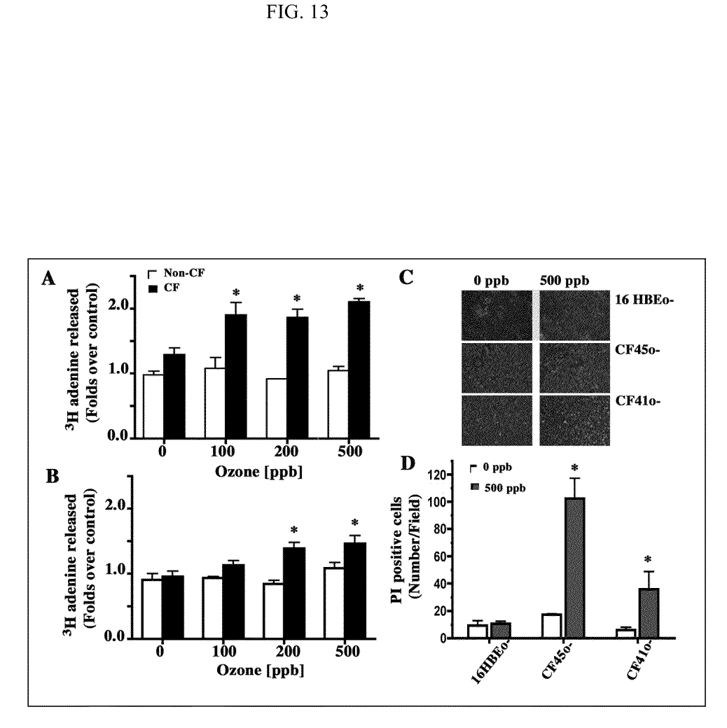

For assessment of membrane damage ALI cultures of airway epithelial cells were labeled with ³H-adenine for 2 h. After 2 h the media was removed, cells were washed (3×) with PBS and exposed to 0, 200 or 500 ppb ozone for 8 h. Apical and basal media was collected and analyzed for ³H-adenine. Cells were harvested for analysis of protein content. Results are shown in FIGS. 13A and 13B. The bars represent means (SEM) (mean of non-CF represents the control) of data of apical media and * indicates significant difference (p<0.05) from 0 ppb.

Exposure to ozone (200 ppb, 24 h) caused a loss of trans epithelial resistance in CF airway epithelial cells whereas the non-CF epithelial cells remained intact (190±68 ohms.cm² in CF4lo- vs. 500±45 ohms.cm² in 16HBEo- cells). Exposure to ozone also for shorter durations (100-500 ppb, 8 h) caused an enhanced loss of membrane integrity in CF airway epithelial cells as indicated by release of tritiated adenine from prelabeled cells (FIGS. 13A and 13B).

Additionally, ALI cultures of non-CF, 16 HBE and CF, CF4lo- and CF45o- cells were exposed to either 0 or 500 ppb ozone for 8 h. At the end of exposure cells were stained with Calcien AM (green, live) and propidium iodide (PI) (red, dead). D) Quantitation of dead PI +ve cells using Image-Pro Plus version 4.0 (Media Cybernetics, Silver Spring, Md.). Results are shown in FIGS. 13C and 13D. The bars represent means (SEM) of data and * indicates significant difference (p<0.05) from 0 ppb.

Exposure of CF airway epithelial cells (CF4lo- and CF45o-) to ozone caused increased uptake of propidium uptake that further reflected membrane damage and cell death (10.0±1.2 in 16HBE vs. 37.0±8.8 PI positive cells per field area in CF4lo-) (FIGS. 13C and 13D). Similar increase in cell death in CF airway epithelial cells was observed using ALI cultures of other pairs of non-CF and CF cell lines and the cells that had biologically inhibited CFTR by antisense CFTR (AS3 cells) oligonucleotides expression (data not shown).

Apoptosis:

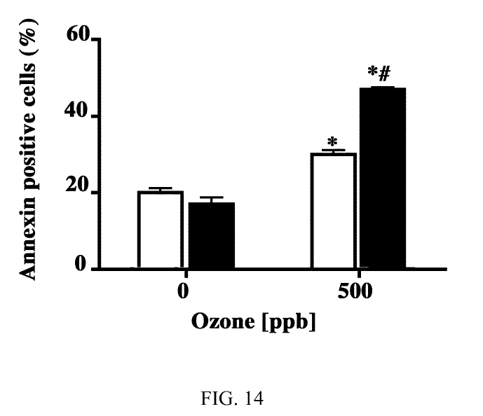

Since uptake of propidium iodide indicated death of cells upon ozone exposure, extent of apoptosis was quantitated using Annexin binding method. ALI cultures of non-CF 16HBEo- and CF CF45o- cells were exposed to either 0 or 500 ppb ozone for 8 h. Cells were collected and cell death was assessed by using Vybrant apotosis assay kit (Molecular Probes, Eugene, Oreg.), as described before. In this method, apoptotic cells bearing phosphatidylserine in the plasma membrane outer leaflet were identified as those binding Alexa Fluor 488-labeled annexin V and propidium iodide was used to assess necrotic cells. Results are summarized in FIG. 14. The bars represent means (SEM) of data and * indicates significant difference (p<0.05) from 0 ppb and # indicates significant difference (p<0.05) from non-CF.

Exposure of non-CF 16HBEo- cells (open bars) cultured at ALI to ozone (500 ppb, 8 h) caused an increase in apoptosis but it was not significantly greater than the 0 ppb exposed cells. In contrast ozone exposure of CF cells CF45o- (closed bars) caused an increased, approximately two fold annexin binding over control (FIG. 14).

Mitochondrial Dysfunction:

CF cells have decreased SERCA2 expression which could modulate intracellular calcium homeostasis (86,95). Both these effects could cause enhanced mitochondrial calcium uptake which upon further exposure to oxidant stress can contribute to mitochondrial dysfunction and eventually cell damage (95-97). Mitochondrial membrane potential (MMP) and cytochrome c release was determined to assess mitochondrial function upon treatment with ozone in non-CF and CF cells.

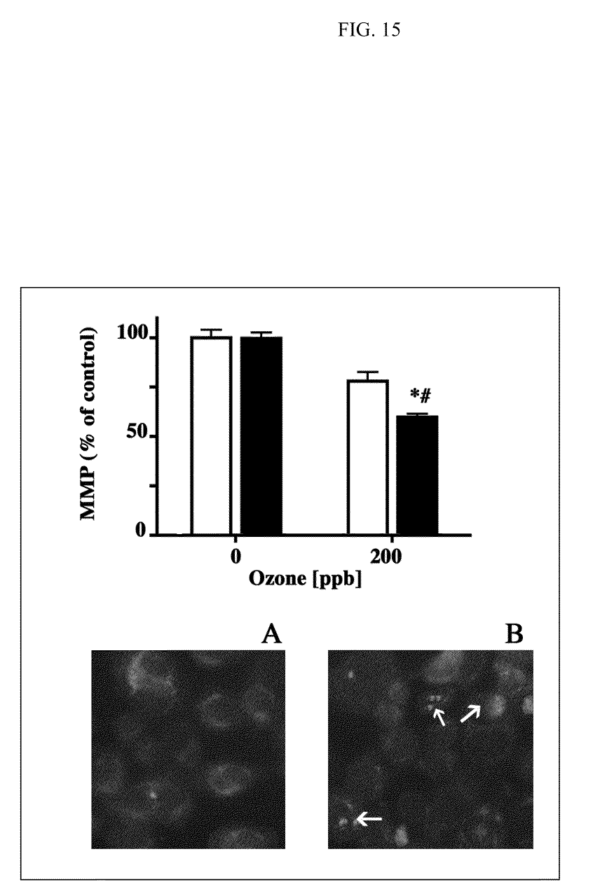

6HBEo- and CF45o- cells were cultured on fibronectin coated 6-well plates and exposed to ozone (500 ppb) for 4 h on the 4^{th} day of plating. Measurement of chloromethyltetramethylrosamine (MitoTracker Orange, Molecular Probes) fluorescence, an indicator of mitochondrial membrane potential (MMP), was performed (90). A mean fluorescence intensity (MFI) of 16HBE cells exposed to 0 ppb ozone was taken as control value. Results are shown in FIG. 15, upper panel. Values are means±SE; n=3 experiments; * Significant difference from 0 ppb control, P<0.05 and # Significant difference from non-CF, P<0.05. Ozone treatment caused a small but not significant decrease in the MMP of the non-CF 16HBEo- (open bars) cells, however in the CF CF4lo- (closed bars) cells there was about 40% decrease in the MMP as compared to the 0 ppb and it was also significantly different from the non-CF 200 ppb exposed cells (FIG. 15 top panel).

Further, cells were fixed and processed for cytochrome c releases using immunocytochemistry. Representative images of 16HBEo- and CF4lo- cells exposed to ozone are shown in FIGS. 15A and 15B. An enhanced cytochrome c release was observed in the CF cells upon ozone exposure (FIG. 15B).

Survival Signaling/ERK1/2 Phosphorylation

Phosphorylation of ERK1/2 is an important component of survival signaling of airway epithelial cells in ozone (81). Whether CF cells have deficient survival signaling response to ozone was investigated next.

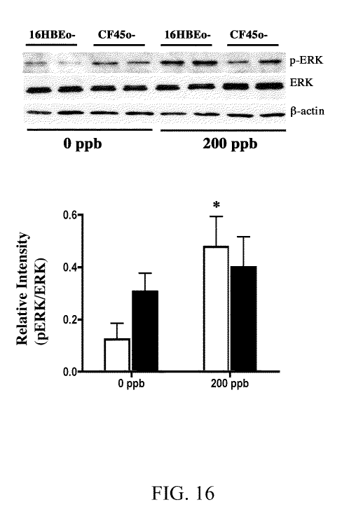

16 HBEo- and CF45o- cells were cultured on fibronectin coated 6-well plates. At about 70-80% confluency, media was removed and replaced with serum free 0.1% BSA containing media. After 24 h cells were exposed to either 0 or 200 ppb ozone for 2 h. Plates were immediately chilled and cells were lysed. The lysates were analyzed for ERK phosphorylation by Western blot using rabbit polyclonal antibodies against ERK1/2 (Upstate Biology) (top panel, FIG. 16). The experiment was performed twice (n=6) and one representative blot is shown. Lower panel, FIG. 16 shows a quantitative estimation of total ERK phosphorylation in non-CF (open bars) and CF cells (closed bars). The bars represent means (SEM) of data and * indicates significant difference (p<0.05) from 0 ppb.

Ozone exposure (200 ppb, 2 h) caused approximately 2.5 folds enhanced phosphorylation of ERK1/2 in non-CF 16HBEo- cells as compared to those exposed to 0 ppb. In contrast the CF, CF4lo- cells ozone-induced ERK1/2 phosphorylation was not significantly different. Interestingly, CF cells had a greater basal ERK1/2 phosphorylation as compared to non-CF cells (FIG. 16).

Exposure to ozone of other non-CF and CF cell line pairs viz. C38 and IB3-1 and S-1 (CFTR sense oligonucleotide transfected 16HBEo- cells, non-CF) and AS3 (CFTR antisense oligonucleotide transfected 16HBEo- cells, CF) cells produced results on cell damage similar to above where CF cells were more susceptible (data not shown). Exposure to 100 or 200 ppb ozone concentrations of differentiated non-CF and CF primary airway epithelial for 8 h did not cause cellular damage that was significantly different from the 0 ppb exposed cells (data not shown).

### Example 14

This example illustrates the Ozone-induced enhanced cytokine release in CF cells.

Ambient concentrations of ozone (50 and 100 ppb, 6 h) may induce airway inflammation through release of proinflammatory mediators from airway epithelial cells (98). The present inventors extended the study of ozone-mediated toxicity in non-CF and CF airway epithelium using close to in vivo models of airways, the differentiated cultures of cells obtained from 6 non-CF and 6 CF donors, to investigate the proinflammatory cytokine release. Three cytokines viz. IL-8, G-CSF and GM-CSF were studied in both apical and basal compartments.

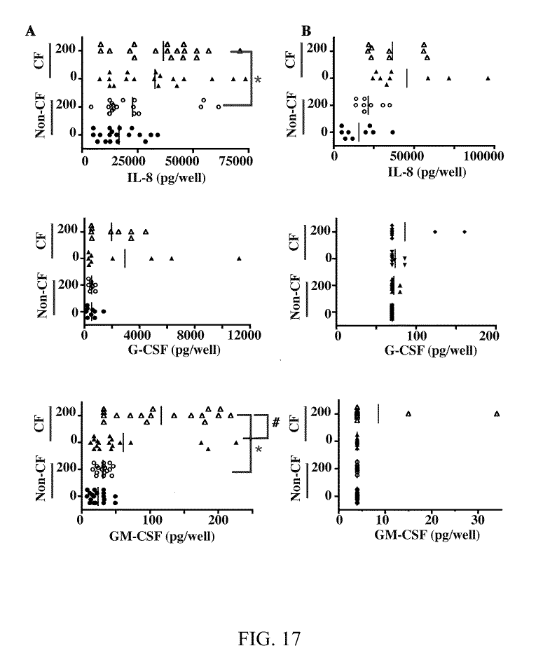

Cells were cultured on collagen coated snapwells and allowed to grow and differentiate for 30 days. Before ozone exposure 100 μl media was added on the apical surface. At the end of exposure (18 h) additional 200 μl media was added apically. After 4 h the media from apical (FIG. 17A) and basolateral (FIG. 17B) surfaces was collected for the analysis of the cytokines Apical and basolateral media was collected, centrifuged and the supernatant were analyzed for cytokines by ELISA at ELISA Tech, Colorado. The results are shown in FIG. 17, differentiated cultures of non-CF (, 0 ppb and ◯, 200 ppb) and CF (▴, 0 ppb and Δ, 200 ppb) primary airway epithelial cells. The bars represent means (SEM) of data and # indicates significant difference (p<0.05) from 0 ppb exposed cells and * indicates significant difference (p<0.05) from 200 ppb non-CF, using Welch's t test.

Although variability among patient samples was relatively high using this cell culture system, the overall pattern indicated that IL-8 was present in both apical and basolateral media (FIG. 17), whereas G-CSF and GM-CSF were at barely detectable levels in the basolateral compartment. In the 200 ppb ozone exposed primary non-CF cells cytokine levels were not significantly different from those that were exposed to 0 ppb. Similarly, in the CF primary airway epithelial cells the levels of Il-8 and G-CSF were not significantly different from 0 ppb. However the GM-CSF values were significantly different in the apical surface of the 200 ppb exposed CF cells. The cytokine values in the apical compartment were significantly increased in CF cells and at 200 ppb they were also significantly enhanced as compared to the non-CF airway epithelial cells (FIG. 17, left panel).

### Example 15

This example illustrates that the Ozone-induced cytokine release in CF cells is NF-κB mediated.

Ozone-mediated cytokine release was studied in polarized air-liquid interface cultures of non-CF and CF cell lines. 16HBEo- and CF4lo- cells were cultured on collagen coated 30 mm inserts. Once cells were polarized (8-10 days in culture) cells were exposed to 0, 100 or 200 ppb ozone with 200 μl media on the apical surface. At the end of exposure (18 h) additional 200 μl media was added to the apical surface. Aliquots were collected after 4 h and analyzed for the cytokine as described before.

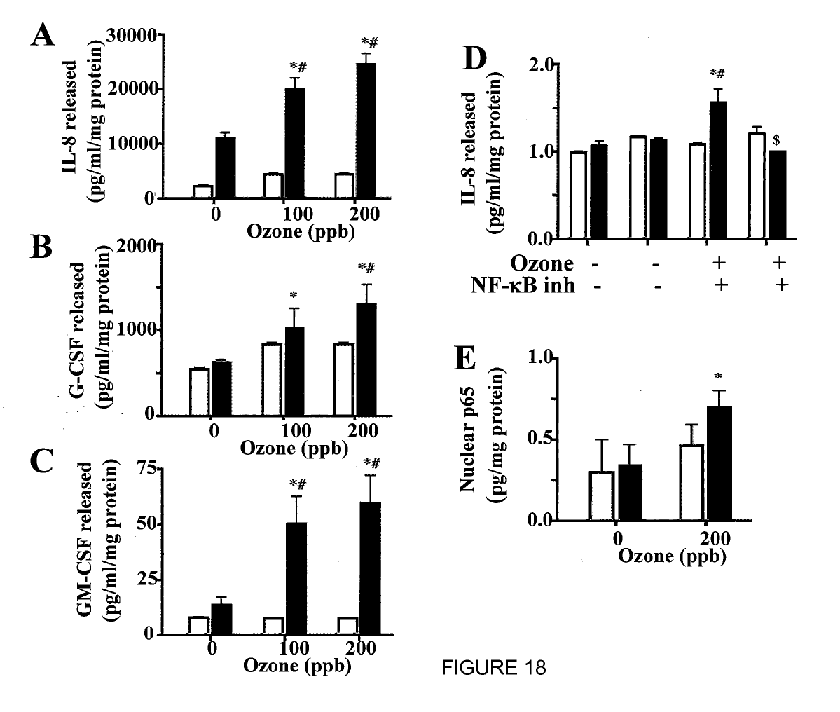

Results are shown in FIG. 18; IL-8 (18A), G-CSF (18B) or GM-CSF (18C). Values are means±SE; n=6, and the figure is a representative of 4 individual experiments; * Significant difference from 0 ppb control, P<0.05 and # Significant difference from non-CF, P<0.05. Effect of preincubation of cells for 30 min with 10 μM [6-amino-4-(4-phenoxyphenylethylamino)quinazoline] (EMD Biosciences, La Jolla, Calif.) (an NF-κB inhibitor) on IL-8 release is shown in FIG. 18D. For analysis of NF-κB activation, nuclear p65 was measured in non-CF and CF cells after exposure to ozone (E). As described above, cells were harvested and nuclear lysates were prepared after exposure to 200 ppb ozone. The cellular fractions were assessed for p65. Values are means±SE; n=6 and the data is a representative of 2 individual experiments; * Significant difference from 0 ppb control, P<0.05 and # Significant difference from non-CF, P<0.05.

Exposure to ozone (100 or 200 ppb, 18 h) caused enhanced IL-8, GM-CSF and G-CSF release in the extracellular media of CF cell line CF45o- as compared to the non-CF 16HBEo- (FIGS. 18A, 18B and 18C). A similar ozone-mediated enhanced cytokine release was observed in other CF cell lines viz, CF4lo-, IB3-1 and AS-3 (data not shown). Preincubation with NF-kB inhibitor caused the IL-8 levels in ozone to drop to control values in the CF cells (FIG. 18D). In these CF cells there was an enhanced p65 mobilization in the nucleus as well (FIG. 18E).

### Example 16

This example illustrates that supplementation with extracellular nucleotides causes increased ozone-mediated cytokine release in CF cells.

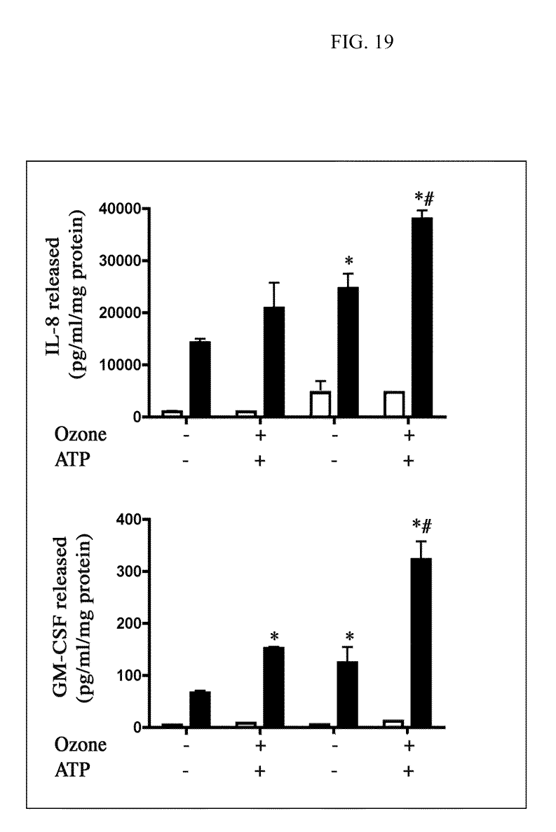

To determine if extracellular nucleotides would be beneficial to the CF cells exposed to ozone, we supplemented with ATP, UTP or INS-45973 (P2Y2 receptor agonist) compound and measured cell death and cytokine release. Non-CF and CF cells were cultured and preincubated with 10 μM ATP for 30 min and then exposed to ozone (200 ppb, 18 h). Apical media was collected and analyzed for IL-8 and GM-CSF. Results are shown in FIG. 19. Values are means±SE; n=6 and the data is a representative of 2 individual experiments; * Significant difference from 0 ppb control, P<0.05 and # Significant difference from non-CF, P<0.05.

Supplementation with extracellular nucleotides (10 or 100 μM) did not prevent cell death due to ozone in the CF cells (data not shown). Also, Preincubating cells with ATP (10 μM) caused an enhanced IL-8 and GM-CSF release in the CF45o- cells (FIG. 19). Similar results were obtained using UTP and INS compound where they enhanced the ozone-mediated cytokine release in CF cells (data not shown). Supplementation of non-CF cells with nucleotides alone did not cause cytokine release.

### Example 17

This example illustrates that SERCA2 modifies ozone-induced cytokine release.

A number of studies have implicated intracellular calcium in the activation of NF-κB and inflammatory signaling responses (99,100).

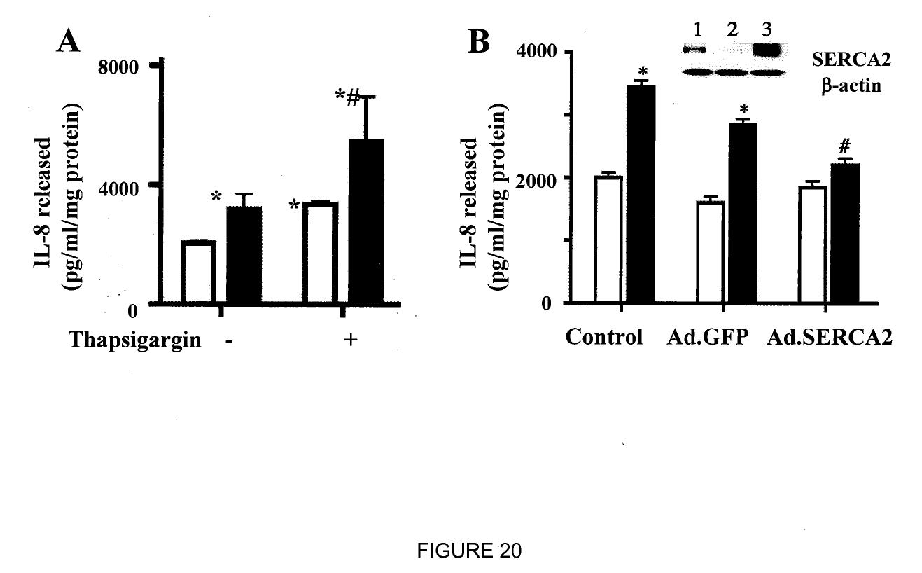

Primary airway epithelial cells were cultured on collagen coated 6-well plates. Cells were preincubated with 2 μM thapsigargin for 30 min and then exposed to 200 ppb ozone for 18 h. The supernatant media was collected and analyzed for cytokines as described before. Results are shown in FIG. 20A. Values are means±SE; n=6 and the data is a representative of 2 individual experiments; * Significant difference from 0 ppb control, P<0.05 and # Significant difference from ozone exposed untreated cells, P<0.05.

Further, cells cultured on 6-well plates were transduced with Ad.GFP or Ad.SERCA2. Transduction of SERCA2 and GFP-encoding adenoviral vectors was carried out as described before. The Ad.SERCA2 was a kind gift of Dr. R J. Hajjar, Harvard Medical School, Massachusetts General Hospital, Cardiovascular Research Center, Charlestown, Ma. The recombinant viruses were added to the cell cultures (Multiplicity of infection, MOI 10:1) on day 3 of culture for 17 hours. The transduction efficiency was estimated by observing green fluorescence of adenoviral GFP-transduced cells. Exposure to ozone (200 ppb, 18 h) was carried out 48 h post transfection. Results are shown in FIG. 20B. The inset is a representative Western blot showing SERCA2 protein overexpression by Ad.SERCA2 in primary airway epithelial cells. Values are means±SE; n=6 and the data is a representative of 2 individual experiments; * Significant difference from 0 ppb control, P<0.05 and # Significant difference from 200 ppb ozone exposed Ad.GFP transduced cells, P<0.05.

Thapsigargin treatment enhanced IL-8 release in control, 0 ppb exposed cells and further augmented ozone mediated IL-8 release (FIG. 20A). Whether SERCA2 overexpression would effect IL-8 release was also determined. In cells transduced with Ad.SERCA2, IL-8 release due to ozone was significantly decreased as compared to the GFP or untransfected controls (FIG. 20B).

### REFERENCES

1. Ratjen F. Recent advances in cystic fibrosis. Paediatr Respir Rev 2008; 9:144-148.
2. Egan M E, Glockner-Pagel J, Ambrose C, Cahill P A, Pappoe L, Balamuth N, Cho E, Canny S, Wagner C A, Geibel J, et al. Calcium-pump inhibitors induce functional surface expression of delta F508-CFTR protein in cystic fibrosis epithelial cells. Nat Med 2002; 8:485-492.
3. Egan M E, Pearson M, Weiner S A, Rajendran V, Rubin D, Glockner-Pagel J, Canny S, Du K, Lukacs G L, Caplan M J. Curcumin, a major constituent of turmeric, corrects cystic fibrosis defects. Science 2004; 304:600-602.
4. Berger A L, Randak C O, Ostedgaard L S, Karp P H, Vermeer D W, Welsh M J. Curcumin stimulates cystic fibrosis transmembrane conductance regulator Cl− channel activity. J Biol Chem 2005; 280:5221-5226.
5. Harada K, Okiyoneda T, Hashimoto Y, Oyokawa K, Nakamura K, Suico M A, Shuto T, Kai H. Curcumin enhances cystic fibrosis transmembrane regulator expression by down-regulating calreticulin. Biochem Biophys Res Commun 2007; 353:351-356.
6. Lipecka J, Norez C, Bensalem N, Baudouin-Legros M, Planelles G, Becq F, Edelman A, Davezac N. Rescue of deltaF508-CFTR (cystic fibrosis transmembrane conductance regulator) by curcumin: Involvement of the keratin 18 network. J Pharmacol Exp Ther 2006; 317:500-505.
7. Norez C, Noel S, Wilke M, Bijvelds M, Jorna H, Melin P, DeJonge H, Becq F. Rescue of functional delF508-CFTR channels in cystic fibrosis epithelial cells by the alpha-glucosidase inhibitor miglustat. FEBS Lett 2006; 580:2081-2086.
8. Wang W, Bernard K, Li G, Kirk K L. Curcumin opens cystic fibrosis transmembrane conductance regulator channels by a novel mechanism that requires neither ATP binding nor dimerization of the nucleotide-binding domains. J Biol Chem 2007; 282:4533-4544.
9. Wang W, Li G, Clancy J P, Kirk K L. Activating cystic fibrosis transmembrane conductance regulator channels with pore blocker analogs. J Biol Chem 2005; 280:23622-23630.
10. Grubb B R, Gabriel S E, Mengos A, Gentzsch M, Randell S H, Van Heeckeren A M, Knowles M R, Drumm M L, Riordan J R, Boucher R C. SERCA pump inhibitors do not correct biosynthetic arrest of deltaF508 CFTR in cystic fibrosis. Am J Respir Cell Mol Biol 2006; 34:355-363.
11. Dragomir A, Bjorstad J, Hjelte L, Roomans G M. Curcumin does not stimulate cAMP-mediated chloride transport in cystic fibrosis airway epithelial cells. Biochem Biophys Res Commun 2004; 322:447-451.
12. Song Y, Sonawane N D, Salinas D, Qian L, Pedemonte N, Galietta L J, Verkman A S. Evidence against the rescue of defective deltaF508-CFTR cellular processing by curcumin in cell culture and mouse models. J Biol Chem 2004; 279:40629-40633.
13. Loo T W, Bartlett M C, Clarke D M. Thapsigargin or curcumin does not promote maturation of processing mutants of the ABC transporters, CFTR, and p-glycoprotein. Biochem Biophys Res Commun 2004; 325:580-585.
14. Ashby M C, Tepikin A V. ER calcium and the functions of intracellular organelles. Semin Cell Dev Biol 2001; 12:11-17.
15. Arai M, Alpert N R, MacLennan D H, Barton P, Periasamy M. Alterations in sarcoplasmic reticulum gene expression in human heart failure. A possible mechanism for alterations in systolic and diastolic properties of the failing myocardium. Circ Res 1993; 72:463-469.
16. Shull G E. Gene knockout studies of Ca²⁺-transporting ATPases. Eur J Biochem 2000; 267:5284-5290.
17. Prasad V, Okunade G W, Miller M L, Shull G E. Phenotypes of SERCA and PMCA knockout mice. Biochem Biophys Res Commun 2004; 322:1192-1203.
18. Schreiber R, Kunzelmann K. Purinergic P2Y6 receptors induce Ca²⁺ and CFTR dependent Cl− secretion in mouse trachea. Cell Physiol Biochem 2005; 16:99-108.
19. Dremina E S, Sharov V S, Kumar K, Zaidi A, Michaelis E K, Schoneich C. Anti-apoptotic protein Bcl-2 interacts with and destabilizes the sarcoplasmic/endoplasmic reticulum Ca²⁺-ATPase (SERCA). Biochem J 2004; 383:361-370.
20. Dremina E S, Sharov V S, Schoneich C. Displacement of SERCA from SR lipid caveolae-related domains by Bcl-2: A possible mechanism for SERCA inactivation. Biochemistry 2006; 45:175-184.
21. Ahmad S, Ahmad A, Guo X, Schneider B K, Jones T, McConville G, Tatreu J, Perraud A, Randell S H, White C W. Differential expression of sarco-endoplasmic reticulum calcium ATPases (SERCAs) in cystic fibrosis (CF) epithelium. Presented at 21st annual North American cystic fibrosis Conference held at Anaheim, Calif. 2007.
22. Ahmad S, Ahmad A, Dremina E S, Sharov V S, Perraud A, Schoneich C, Randell S H, White C W. Bcl-2 suppresses SERCA2 expression in cystic fibrosis airway epithelial cells. Presented at Lung Epithelium and Disease, FASEB Summer Research Conference held at Saxtons Rivers, Vt. 2008.
23. Fulcher M L, Gabriel S, Burns K A, Yankaskas J R, Randell S H. Well-differentiated human airway epithelial cell cultures. Methods Mol Med 2005; 107:183-206.
24. Kube D, Sontich U, Fletcher D, Davis P B. Proinflammatory cytokine responses to P. aeruginosa infection in human airway epithelial cell lines. Am J Physiol Lung Cell Mol Physiol 2001; 280:L493-502.
25. Fulcher M L, Gabriel S E, Olsen J C, Tatreau J R, Gentzsch M, Livanos E, Saavedra M T, Salmon P, Randell S H. Novel human bronchial epithelial cell lines for cystic fibrosis research. Am J Physiol Lung Cell Mol Physiol 2009; 296:L82-91.
26. Ahmad A, Ahmad S, Chang L Y, Schaack J, White C W. Endothelial Akt activation by hyperoxia: Role in cell survival. Free Radic Biol Med 2006; 40:1108-1118.
27. Vais H, Gao G P, Yang M, Tran P, Louboutin J P, Somanathan S, Wilson J M, Reenstra W W. Novel adenoviral vectors coding for GFP-tagged wtCFTR and deltaF508-CFTR: Characterization of expression and electrophysiological properties in A549 cells. Pflugers Arch 2004; 449:278-287.
28. Park J, Fang S, Crews A L, Lin K W, Adler K B. MARCKS regulation of mucin secretion by airway epithelium in vitro: Interaction with chaperones. Am J Respir Cell Mol Biol 2008.
29. Arnaudeau S, Frieden M, Nakamura K, Castelbou C, Michalak M, Demaurex N. Calreticulin differentially modulates calcium uptake and release in the endoplasmic reticulum and mitochondria. J Biol Chem 2002; 277:46696-46705.
30. Wu R, Zhao Y H, Chang M M. Growth and differentiation of conducting airway epithelial cells in culture. Eur Respir J 1997; 10:2398-2403.
31. Ostedgaard L S, Rogers C S, Dong Q, Randak C O, Vermeer D W, Rokhlina T, Karp P H, Welsh M J. Processing and function of CFTR-deltaF508 are species-dependent. Proc Natl Acad Sci USA 2007; 104:15370-15375.
32. Kuo T H, Kim H R, Zhu L, Yu Y, Lin H M, Tsang W. Modulation of endoplasmic reticulum calcium pump by Bcl-2. Oncogene 1998; 17:1903-1910.
33. Weber A J, Soong G, Bryan R, Saba S, Prince A. Activation of NF-kappaB in airway epithelial cells is dependent on CFTR trafficking and Cl− channel function. Am J Physiol Lung Cell Mol Physiol 2001; 281:L71-78.
34. Machen T E. Innate immune response in CF airway epithelia: Hyperinflammatory? Am J Physiol Cell Physiol 2006; 291:C218-230.
35. Wang C Y, Guttridge D C, Mayo M W, Baldwin A S, Jr. NF-kappaB induces expression of the Bcl-2 homologue A1/bfl-1 to preferentially suppress chemotherapy-induced apoptosis. Mol Cell Biol 1999; 19:5923-5929.
36. Liu X, Togo S, Al-Mugotir M, Kim H, Fang Q, Kobayashi T, Wang X, Mao L, Bitterman P, Rennard S, NF-kappaB mediates the survival of human bronchial epithelial cells exposed to cigarette smoke extract. Respir Res 2008; 9:66.
37. Osika E, Cavaillon J M, Chadelat K, Boule M, Fitting C, Tournier G, Clement A. Distinct sputum cytokine profiles in cystic fibrosis and other chronic inflammatory airway disease. Eur Respir J 1999; 14:339-346.
38. Vasanji Z, Dhalla N S, Netticadan T. Increased inhibition of SERCA2 by phospholamban in the type I diabetic heart. Mol Cell Biochem 2004; 261:245-249.
39. Randriamboavonjy V, Pistrosch F, Bolck B, Schwinger R H, Dixit M, Badenhoop K, Cohen R A, Busse R, Fleming I. Platelet sarcoplasmic endoplasmic reticulum Ca²⁺-atpase and mu-calpain activity are altered in type 2 diabetes mellitus and restored by rosiglitazone. Circulation 2008; 117:52-60.
40. Ribeiro C M, Paradiso A M, Carew M A, Shears S B, Boucher R C. Cystic fibrosis airway epithelial Ca²⁺ᵢ signaling: The mechanism for the larger agonist-mediated Ca²⁺ᵢ signals in human cystic fibrosis airway epithelia. J Biol Chem 2005; 280:10202-10209.
41. Ribeiro C M. The role of intracellular calcium signals in inflammatory responses of polarised cystic fibrosis human airway epithelia. Drugs R D 2006; 7:17-31.
42. Harris J F, Fischer M J, Hotchkiss J R, Monia B P, Randell S H, Harkema J R, Tesfaigzi Y. Bcl-2 sustains increased mucous and epithelial cell numbers in metaplastic airway epithelium. Am J Respir Crit. Care Med 2005; 171:764-772.
43. Nichols D, Chmiel J, Berger M. Chronic inflammation in the cystic fibrosis lung: Alterations in inter- and intracellular signaling. Clin Rev Allergy Immunol 2008; 34:146-162.
44. Heckman C A, Mehew J W, Boxer L M. NF-kappaB activates Bcl-2 expression in t(14;18) lymphoma cells. Oncogene 2002; 21:3898-3908.
45. Voynow J A, Fischer B M, Roberts B C, Proia A D. Basal-like cells constitute the proliferating cell population in cystic fibrosis airways. Am J Respir Crit. Care Med 2005; 172:1013-1018.
46. Braunstein G M, Roman R M, Clancy J P, Kudlow B A, Taylor A L, Shylonsky V G, Jovov B, Peter K, Jilling T, Ismailov, I I, et al. Cystic fibrosis transmembrane conductance regulator facilitates ATP release by stimulating a separate ATP release channel for autocrine control of cell volume regulation. J Biol Chem 2001; 276:6621-6630.
47. Braunstein G M, Zsembery A, Tucker T A, Schwiebert E M. Purinergic signaling underlies CFTR control of human airway epithelial cell volume. J Cyst Fibros 2004; 3:99-117.
48. Shen M R, Yang T P, Tang M J. A novel function of Bcl-2 overexpression in regulatory volume decrease. Enhancing swelling-activated Ca(2+) entry and Cl(−) channel activity. J Biol Chem 2002; 277:15592-15599.
49. Portier B P, Taglialatela G. Bcl-2 localized at the nuclear compartment induces apoptosis after transient overexpression. J Biol Chem 2006; 281:40493-40502.
50. Li C, Wang X, Vais H, Thompson C B, Foskett J K, White C. Apoptosis regulation by Bcl-x(l) modulation of mammalian inositol 1,4,5-trisphosphate receptor channel isoform gating. Proc Natl Acad Sci USA 2007; 104:12565-12570.
51. Granville D J, Ruehlmann D O, Choy J C, Cassidy B A, Hunt D W, van Breemen C, McManus BM. Bcl-2 increases emptying of endoplasmic reticulum Ca²⁺ stores during photodynamic therapy-induced apoptosis. Cell Calcium 2001; 30:343-350.
52. Zhou Y P, Teng D, Dralyuk F, Ostrega D, Roe M W, Philipson L, Polonsky K S. Apoptosis in insulin-secreting cells. Evidence for the role of intracellular Ca²⁺ stores and arachidonic acid metabolism. J Clin Invest 1998; 101:1623-1632.
53. Xu C, Xu W, Palmer A E, Reed J C. Bi-1 regulates endoplasmic reticulum Ca²⁺ homeostasis downstream of Bcl-2 family proteins. J Biol Chem 2008; 283:11477-11484.
54. Rottner M, Kunzelmann C, Mergey M, Freyssinet J M, Martinez M C. Exaggerated apoptosis and NF-kappaB activation in pancreatic and tracheal cystic fibrosis cells. FASEB J 2007; 21:2939-2948.
55. Ornatowski W, Poschet J F, Perkett E, Taylor-Cousar J L, Deretic V. Elevated furin levels in human cystic fibrosis cells result in hypersusceptibility to exotoxin a-induced cytotoxicity. J Clin Invest 2007; 117:3489-3497.
56. Krunkosky T M, Fischer B M, Martin L D, Jones N, Akley N J, Adler K B. Effects of TNF-alpha on expression of ICAM-1 in human airway epithelial cells in vitro. Signaling pathways controlling surface and gene expression. Am J Respir Cell Mol Biol 2000; 22:685-692.
57. Ying J, Sharov V, Xu S, Jiang B, Gerrity R, Schoneich C, Cohen R A. Cysteine-674 oxidation and degradation of sarcoplasmic reticulum Ca(2+) ATPase in diabetic pig aorta. Free Radic Biol Med 2008; 45 :756-762.
58. Vangheluwe P, Raeymaekers L, Dode L, Wuytack F. Modulating sarco(endo)plasmic reticulum Ca²⁺ ATPase2 (SERCA2) activity: Cell biological implications. Cell Calcium 2005; 38:291-302.
59. White N M, Jiang D, Burgess J D, Bederman I R, Previs S F, Kelley T J. Altered cholesterol homeostasis in cultured and in vivo models of cystic fibrosis. Am J Physiol Lung Cell Mol Physiol 2007; 292:L476-486.
60. Talukder M A, Kalyanasundaram A, Zuo L, Velayutham M, Nishijima Y, Periasamy M, Zweier J L. Is reduced SERCA2a expression detrimental or beneficial to postischemic cardiac function and injury? Evidence from heterozygous SERCA2a knockout mice. Am J Physiol Heart Circ Physiol 2008; 294:H1426-1434.
61. Deterding R R, Lavange L M, Engels J M, Mathews D W, Coquillette S J, Brody A S, Millard S P, Ramsey B W. Phase 2 randomized safety and efficacy trial of nebulized denufosol tetrasodium in cystic fibrosis. Am J Respir Crit. Care Med 2007; 176:362-369.
62. Blouquit S, Regnier A, Dannhoffer L, Fermanian C, Naline E, Boucher R, Chinet T. Ion and fluid transport properties of small airways in cystic fibrosis. Am J Respir Crit. Care Med 2006; 174:299-305.
63. Ahmad S, Ahmad A, McConville G, Schneider B K, Allen C B, Manzer R, Mason R J, White C W. Lung epithelial cells release ATP during ozone exposure: Signaling for cell survival. Free Radic Biol Med 2005; 39:213-226.
64. Ahmad S, Ahmad A, Ghosh M, Leslie C C, White C W. Extracellular ATP-mediated signaling for survival in hyperoxia-induced oxidative stress. J Biol Chem 2004; 279:16317-16325.
65. Dremina E S, Sharov V S, Kumar K, Zaidi A, Michaelis E K, Schoneich C. Anti-apoptotic protein bcl-2 interacts with and destabilizes the sarcoplasmic/endoplasmic reticulum Ca²⁺-ATPase (SERCA). Biochem J 2004; 383:361-370.
66. Riddle S R, Ahmad A, Ahmad S, Deeb S S, Malkki M, Schneider B K, Allen C B, White C W. Hypoxia induces hexokinase ii gene expression in human lung cell line A549. Am J Physiol Lung Cell Mol Physiol 2000; 278:L407-416.
67. Wootton L L, Argent C C, Wheatley M, Michelangeli F. The expression, activity and localisation of the secretory pathway Ca²⁺-ATPase (SPCA1) in different mammalian tissues. Biochim Biophys Acta 2004; 1664:189-197.
68. Michelangeli F, Munkonge F M. Methods of reconstitution of the purified sarcoplasmic reticulum (Ca(²⁺)—Mg²⁺)-ATPase using bile salt detergents to form membranes of defined lipid to protein ratios or sealed vesicles. Anal Biochem 1991; 194:231-236.
69. Dremina E S, Sharov V S, Schoneich C. Displacement of SERCA from SR lipid caveolae-related domains by bcl-2: A possible mechanism for SERCA inactivation. Biochemistry 2006; 45:175-184.
70. Li C, Duan W, Yang F, Zhang X. Caveolin-3-anchored microdomains at the rabbit sarcoplasmic reticulum membranes. Biochem Biophys Res Commun 2006; 344:1135-1140.
71. Goss, C. H., Newsom, S. A., Schildcrout, J. S., Sheppard, L., and Kaufman, J. D. (2004) Am J Respir Crit. Care Med 169, 816-821
72. Rabinovitch, N., Strand, M., and Gelfand, E. W. (2006) Am J Respir Crit. Care Med 173, 1098-1105
73. Gilliland, F. D. (2009) Pediatrics 123 Suppl 3, S168-173
74. Harkema, J. R., Plopper, C. G., Hyde, D. M., St George, J. A., Wilson, D. W., and Dungworth, D. L. (1993) Am J Pathol 143, 857-866
75. Bhalla, D. K. (1999) J Toxicol Environ Health B Crit. Rev 2, 31-86
76. Cho, H. Y., Hotchkiss, J. A., Bennett, C. B., and Harkema, J. R. (2000) Am J Respir Crit. Care Med 162, 629-636
77. Damera, G., Zhao, H., Wang, M., Smith, M., Kirby, C., Jester, W. F., Lawson, J. A., and Panettieri, R. A., Jr. (2009) Am J Physiol Lung Cell Mol Physiol 296, L674-683
78. Ahmad, S., Ahmad, A., McConville, G., Schneider, B. K., Allen, C. B., Manzer, R., Mason, R. J., and White, C. W. (2005) Free Radic Biol Med 39, 213-226
79. Ahmad, S., Ahmad, A., Ghosh, M., Leslie, C. C., and White, C. W. (2004) J Biol Chem 279, 16317-16325
80. Braunstein, G. M., Roman, R. M., Clancy, J. P., Kudlow, B. A., Taylor, A. L., Shylonsky, V. G., Jovov, B., Peter, K., Jilling, T., Ismailov, I I, Benos, D. J., Schwiebert, L. M., Fitz, J. G., and Schwiebert, E. M. (2001) J Biol Chem 276, 6621-6630
81. Ahmad, S., Ahmad, A., and White, C. W. (2006) Free Radic Biol Med 41, 29-40
82. Palmer, M. L., Lee, S. Y., Maniak, P. J., Carlson, D., Fahrenkrug, S. C., and O'Grady, S. M. (2006) Am J Physiol Cell Physiol 290, C1189-1198
83. Chappe, V., Hinkson, D. A., Howell, L. D., Evagelidis, A., Liao, J., Chang, X. B., Riordan, J. R., and Hanrahan, J. W. (2004) Proc Natl Acad Sci USA 101, 390-395
84. Boucher, R. C., Cheng, E. H., Paradiso, A. M., Stutts, M. J., Knowles, M. R., and Earp, H. S. (1989) J Clin Invest 84, 1424-1431
85. Graham, A., Steel, D. M., Alton, E. W., and Geddes, D. M. (1992) J Physiol 453, 475-491
86. Ahmad, S., Ahmad, A., Dremina, E. S., Sharov, V. S., Guo, X., Jones, T. N., Loader, J. E., Tatreau, J. R., Perraud, A. L., Schoneich, C., Randell, S. H., and White, C. W. (2009) Am J Respir Crit. Care Med 179, 816-826
87. Fulcher, M. L., Gabriel, S., Burns, K. A., Yankaskas, J. R., and Randell, S. H. (2005) Methods Mol Med 107, 183-206
88. Kube, D., Sontich, U., Fletcher, D., and Davis, P. B. (2001) Am J Physiol Lung Cell Mol Physiol 280, L493-502
89. Jefferson, D. M., Valentich, J. D., Marini, F. C., Grubman, S. A., Iannuzzi, M. C., Dorkin, H. L., Li, M., Klinger, K. W., and Welsh, M. J. (1990) Am J Physiol 259, L496-505
90. Ahmad, S., White, C. W., Chang, L. Y., Schneider, B. K., and Allen, C. B. (2001) Am J Physiol Lung Cell Mol Physiol 280, L779-791
91. Ahmad, A., Ahmad, S., Chang, L. Y., Schaack, J., and White, C. W. (2006) Free Radic Biol Med 40, 1108-1118
92. Reigada, D., and Mitchell, C. H. (2005) Am J Physiol Cell Physiol 288, C132-140
93. Qu, F., Qin, X. Q., Cui, Y. R., Xiang, Y., Tan, Y. R., Liu, H. J., Peng, L. H., Zhou, X. Y., Liu, C., and Zhu, X. L. (2009) Chem Biol Interact 179, 219-226
94. Reid, D. W., Misso, N., Aggarwal, S., Thompson, P. J., and Walters, E. H. (2007) Respirology 12, 63-69
95. Ji, Y., Lalli, M. J., Babu, G. J., Xu, Y., Kirkpatrick, D. L., Liu, L. H., Chiamvimonvat, N., Walsh, R. A., Shull, G. E., and Periasamy, M. (2000) J Biol Chem 275, 38073-38080
96. Aydin, J., Andersson, D. C., Hanninen, S. L., Wredenberg, A., Tavi, P., Park, C. B., Larsson, N. G., Bruton, J. D., and Westerblad, H. (2009) Hum Mol Genet. 18, 278-288
97. Lattanzio, F. A., Jr., Tiangco, D., Osgood, C., Beebe, S., Kerry, J., and Hargrave, B. Y. (2005) Cardiovasc Toxicol 5, 377-390
98. Rusznak, C., Devalia, J. L., Sapsford, R. J., and Davies, R. J. (1996) Eur Respir J 9, 2298-2305
99. Fu, Z., Bettega, K., Carroll, S., Buchholz, K. R., and Machen, T. E. (2007) Am J Physiol Lung Cell Mol Physiol 292, L353-364
100. Hybiske, K., Fu, Z., Schwarzer, C., Tseng, J., Do, J., Huang, N., and Machen, T. E. (2007) Am J Physiol Lung Cell Mol Physiol 293, L1250-1260
101. Uhlson, C., Harrison, K., Allen, C. B., Ahmad, S., White, C. W., and Murphy, R. C. (2002) Chem Res Toxicol 15, 896-906
102. Wesley, U. V., Bove, P. F., Hristova, M., McCarthy, S., and van der Vliet, A. (2007) J Biol Chem 282, 3213-3220
103. Cobb, B. R., Fan, L., Kovacs, T. E., Sorscher, E. J., and Clancy, J. P. (2003) Am J Respir Cell Mol Biol 29, 410-418
104. Bucheimer, R. E., and Linden, J. (2004) J Physiol 555, 311-321
105. Braunstein, G. M., Zsembery, A., Tucker, T. A., and Schwiebert, E. M. (2004) J Cyst Fibros 3, 99-117
106. Lazarowski, E. R., Tarran, R., Grubb, B. R., van Heusden, C. A., Okada, S., and Boucher, R. C. (2004) J Biol Chem 279, 36855-36864
107. Perez, A., Issler, A. C., Cotton, C. U., Kelley, T. J., Verkman, A. S., and Davis, P. B. (2007) Am J Physiol Lung Cell Mol Physiol 292, L383-395
108. Barriere, H., Poujeol, C., Tauc, M., Blasi, J. M., Counillon, L., and Poujeol, P. (2001) Am J Physiol Cell Physiol 281, C810-824
109. Jungas, T., Motta, I., Duffieux, F., Fanen, P., Stoven, V., and Ojcius, D. M. (2002) J Biol Chem 277, 27912-27918
110. Maiuri, L., Raia, V., De Marco, G., Coletta, S., de Ritis, G., Londei, M., and Auricchio, S. (1997) FEBS Lett 408, 225-231
111. Harkema, J. R., and Hotchkiss, J. A. (1993) Toxicol Lett 68, 251-263
112. Sathishkumar, K., Gao, X., Raghavamenon, A. C., Parinandi, N., Pryor, W. A., and Uppu, R. M. (2009) Free Radic Biol Med 47, 548-558
113. Oslund, K. L., Hyde, D. M., Putney, L. F., Alfaro, M. F., Walby, W. F., Tyler, N. K., and Schelegle, E. S. (2008) Am J Respir Cell Mol Biol 39, 279-288
114. Verhaeghe, C., Tabruyn, S. P., Oury, C., Bours, V., and Griffioen, A. W. (2007) Biochem Biophys Res Commun 356, 745-749
115. Verhaeghe, C., Remouchamps, C., Hennuy, B., Vanderplasschen, A., Chariot, A., Tabruyn, S. P., Oury, C., and Bours, V. (2007) Biochem Pharmacol 73, 1982-1994
116. Bosson, J., Stenfors, N., Bucht, A., Helleday, R., Pourazar, J., Holgate, S. T., Kelly, F. J., Sandstrom, T., Wilson, S., Frew, A. J., and Blomberg, A. (2003) Clin Exp Allergy 33, 777-782
117. Aldallal, N., McNaughton, E. E., Manzel, L. J., Richards, A. M., Zabner, J., Ferkol, T. W., and Look, D. C. (2002) Am J Respir Crit. Care Med 166, 1248-1256
118. DiMango, E., Ratner, A. J., Bryan, R., Tabibi, S., and Prince, A. (1998) J Clin Invest 101, 2598-2605
119. Venkatakrishnan, A., Stecenko, A. A., King, G., Blackwell, T. R., Brigham, K. L., Christman, J. W., and Blackwell, T. S. (2000) Am J Respir Cell Mol Biol 23, 396-403
120. Ratner, A. J., Bryan, R., Weber, A., Nguyen, S., Barnes, D., Pitt, A., Gelber, S., Cheung, A., and Prince, A. (2001) J Biol Chem 276, 19267-19275
121. Meehan, S., Wu, A. J., Kang, E. C., Sakai, T., and Ambudkar, I. S. (1997) Am J Physiol 273, C2030-2036
122. Sathish, V., Thompson, M. A., Bailey, J. P., Pabelick, C. M., Prakash, Y. S., and Sieck, G. C. (2009) Am J Physiol Lung Cell Mol Physiol 297, L26-34

What is claimed is:

1. A method to treat a pulmonary disease in a subject, comprising increasing the biological activity of Sarcoendoplasmic Reticulum Calcium ATPase 2 (SERCA2) protein in the cells of the subject.

2. The method of claim 1, wherein the SERCA2 protein is expressed in airway epithelial cells.

3. The method of claim 1, comprising administering the subject with an effective amount of an agent that increases the biological activity of the SERCA2 protein.

4. The method of claim 3, wherein the agent comprises a) SERCA2 protein or a homologue thereof, b) a compound that increases the expression of the SERCA2 protein, or c) a SERCA2 activator compound that increases the biological activity of the SERCA2 protein.

5. The method of claim 4, wherein the SERCA2 protein or a homologue thereof is recombinantly produced.

6. The method of claim 4, wherein the compound that increases the expression of the SERCA2 protein comprises a recombinant nucleic acid molecule encoding the SERCA2 protein or a homologue thereof.

7. The method of claim 4, wherein the SERCA2 activator compound comprises PST2744 [Istaroxime; (E,Z)-3-((2-aminoethoxy)imino) androstane-6,17-dione hydrochloride)], Memnopeptide A, JTV-519, CDN1054, albuterol, xopenex, IGF (insulin like growth factor), EGF (epithelial growth factor), or rosiglitazone.

8. The method of claim 1, wherein the pulmonary disease is cystic fibrosis.

9. The method of claim 3, wherein the agent comprises a pharmaceutically acceptable carrier.

10. The method of claim 3, wherein the step of administering comprises providing the agent as a tablet, a powder, an effervescent tablet, an effervescent powder, a capsule, a liquid, a suspension, a granule or a syrup.

11. The method of claim 1, wherein said subject is a human.

12. A method to protect a subject from exposure to an oxidizing gas, comprising increasing the biological activity of Sarcoendoplasmic Reticulum Calcium ATPase 2 (SERCA2) protein in the cells of the subject.

13. The method of claim 12, wherein the subject has an a pulmonary disease and wherein exposure to oxidizing gas leads to enhanced airway epithelial cell death and inflammation leading to exacerbation of the pulmonary disease.

14. The method of claim 12, wherein the gas comprises ozone, oxygen, chlorine or mustard gas.

15. The method of claim 12, comprising administering to the subject with an effective amount of an agent that increases the biological activity of the SERCA2 protein, wherein the agent comprises a) SERCA2 protein or a homologue thereof, b) a compound that increases the expression of the SERCA2 protein, or c) a SERCA2 activator compound that increases the biological activity of the SERCA2 protein.

16. A method for diagnosing a pulmonary disease comprising:
a) detecting a level of expression or biological activity of the SERCA2 protein in a test sample; and b) comparing the level of expression or biological activity of the SERCA2 protein in the test sample to a baseline level of SERCA2 protein expression or activity established from a control sample; wherein detection of a statistically significant difference in the SERCA2 protein expression or biological activity in the test sample, as compared to the baseline level of SERCA2 protein expression or biological activity, is an indicator of the presence of the pulmonary disease or the potential therefor in the test sample as compared to cells in the control sample.

17. The method of claim 16, wherein the detecting the level of expression or biological activity of the SERCA2 protein in a sample comprises detecting SERCA2 mRNA in the sample, or detecting SERCA2 protein in the sample, or detecting SERCA2 protein biological activity in the sample.

18. A method to evaluate the efficacy of a treatment of a pulmonary disease in a subject, comprising
a) detecting the level of expression or biological activity of SERCA2 in a test sample taken from the subject before administering the treatment;
b) detecting the level of expression or biological activity of SERCA2 in a test sample taken from the subject after administering the treatment;
c) comparing the level of the expression or biological activity of the SERCA2 from step (a) in the test sample taken from the subject before administering the treatment to the level of the expression or biological activity of the SERCA2 from step (b) in the test sample taken from the subject after administering the treatment.

19. The method of claim 18, wherein detecting the level of expression or biological activity of SERCA2 in a test sample comprises detecting SERCA2 mRNA in the test sample, or detecting SERCA2 protein in the test sample, or detecting SERCA2 protein biological activity in the test sample.

20. The method of claim 16 or 18, wherein the pulmonary disease is Cystic Fibrosis.

21. The method of claim 6, wherein the recombinant nucleic acid molecule encoding the SERCA2 protein or a homologue thereof comprises a sequence selected from the group consisting of: NM_{—}170665.3 or GI:161377445, NM_{—}001681.3 or GI:161377446, and NM_{—}001135765.1 or GI:209413708.

22. The method of claim 4, wherein the SERCA2 protein or a homologue thereof comprises an amino acid sequence selected from the group consisting of NP_{—}733765.1 or GI:24638454, NP_{—}001672.1 or GI:4502285, and NP_{—}001129237.1, or GI:209413709.
